SEQUENCE LISTING (99 sequences)

SEQ ID NO: 1 - DNA, 30 bp, Artificial Sequence
  Description of Artificial Sequence Synthetic oligonucleotide
tgactggatccgcatgacattcgccgtaag

SEQ ID NO: 2 - DNA, 30 bp, Artificial Sequence
  Description of Artificial Sequence Synthetic oligonucleotide
gtcatggatccgcatgacattcgccgtaag

SEQ ID NO: 3 - DNA, 30 bp, Artificial Sequence
  Description of Artificial Sequence Synthetic oligonucleotide
atcgtggatccgcatgacattcgccgtaag

SEQ ID NO: 4 - DNA, 30 bp, Artificial Sequence
  Description of Artificial Sequence Synthetic oligonucleotide
atgctggatccgcatgacattcgccgtaag

SEQ ID NO: 5 - DNA, 30 bp, Artificial Sequence
  Description of Artificial Sequence Synthetic oligonucleotide
gcaatggatccgcatgacattcgccgtaag

SEQ ID NO: 6 - DNA, 30 bp, Artificial Sequence
  Description of Artificial Sequence Synthetic oligonucleotide
tgaccttacggcgaatgaccgaatcagcct

SEQ ID NO: 7 - DNA, 30 bp, Artificial Sequence
  Description of Artificial Sequence Synthetic oligonucleotide
cgatcttacggcgaatgaccgaatcagcct

SEQ ID NO: 8 - DNA, 30 bp, Artificial Sequence
  Description of Artificial Sequence Synthetic oligonucleotide
gcatcttacggcgaatgaccgaatcagcct

SEQ ID NO: 9 - DNA, 30 bp, Artificial Sequence
  Description of Artificial Sequence Synthetic oligonucleotide
ttgccttacggcgaatgaccgaatcagcct

SEQ ID NO: 10 - DNA, 30 bp, Artificial Sequence
  Description of Artificial Sequence Synthetic oligonucleotide
ggatcttacggcgaatgaccgaatcagcct

SEQ ID NO: 11 - DNA, 30 bp, Artificial Sequence
  Description of Artificial Sequence Synthetic oligonucleotide
tgacaggctgattcggttcatgcggatcca

SEQ ID NO: 12 - DNA, 30 bp, Artificial Sequence
  Description of Artificial Sequence Synthetic oligonucleotide
cgataggctgattcggttcatgcggatcca

SEQ ID NO: 13 - DNA, 30 bp, Artificial Sequence
  Description of Artificial Sequence Synthetic oligonucleotide
gcataggctgattcggttcatgcggatcca

SEQ ID NO: 14 - DNA, 30 bp, Artificial Sequence
  Description of Artificial Sequence Synthetic oligonucleotide
ttgcaggctgattcggttcatgcggatcca

SEQ ID NO: 15 - DNA, 30 bp, Artificial Sequence
  Description of Artificial Sequence Synthetic oligonucleotide
ggataggctgattcggttcatgcggatcca

SEQ ID NO: 16 - DNA, 4 bp, Artificial Sequence
  Description of Artificial Sequence Synthetic oligonucleotide
tgac

SEQ ID NO: 17 - DNA, 4 bp, Artificial Sequence
  Description of Artificial Sequence Synthetic oligonucleotide
gtca

SEQ ID NO: 18 - DNA, 4 bp, Artificial Sequence
  Description of Artificial Sequence Synthetic oligonucleotide
cgat

SEQ ID NO: 19 - DNA, 4 bp, Artificial Sequence
  Description of Artificial Sequence Synthetic oligonucleotide
atcg

SEQ ID NO: 20 - DNA, 4 bp, Artificial Sequence
  Description of Artificial Sequence Synthetic oligonucleotide
gcat

SEQ ID NO: 21 - DNA, 4 bp, Artificial Sequence
  Description of Artificial Sequence Synthetic oligonucleotide
atgc

SEQ ID NO: 22 - DNA, 4 bp, Artificial Sequence
  Description of Artificial Sequence Synthetic oligonucleotide
ttgc

SEQ ID NO: 23 - DNA, 4 bp, Artificial Sequence
  Description of Artificial Sequence Synthetic oligonucleotide
gcaa

SEQ ID NO: 24 - DNA, 4 bp, Artificial Sequence
  Description of Artificial Sequence Synthetic oligonucleotide
ggat

SEQ ID NO: 25 - DNA, 13 bp, Artificial Sequence
  Description of Artificial Sequence Synthetic oligonucleotide
tggatccgcatga

SEQ ID NO: 26 - DNA, 13 bp, Artificial Sequence
  Description of Artificial Sequence Synthetic oligonucleotide
cattcgccgtaag

SEQ ID NO: 27 - DNA, 13 bp, Artificial Sequence
  Description of Artificial Sequence Synthetic oligonucleotide
cttacggcgaatg

SEQ ID NO: 28 - DNA, 13 bp, Artificial Sequence
  Description of Artificial Sequence Synthetic oligonucleotide
accgaatcagcct

SEQ ID NO: 29 - DNA, 13 bp, Artificial Sequence
  Description of Artificial Sequence Synthetic oligonucleotide
aggctgattcggt

SEQ ID NO: 30 - DNA, 13 bp, Artificial Sequence
  Description of Artificial Sequence Synthetic oligonucleotide
tcatgcggatcca

SEQ ID NO: 31 - DNA, 39 bp, Artificial Sequence
  Description of Artificial Sequence Synthetic oligonucleotide
actgctggatcgtatgcgtagtctggacgtctaccgtgt

SEQ ID NO: 32 - DNA, 26 bp, Artificial Sequence
  Description of Artificial Sequence Synthetic oligonucleotide
cagtgcaggctacgcataccatccag

SEQ ID NO: 33 - DNA, 26 bp, Artificial Sequence
  Description of Artificial Sequence Synthetic oligonucleotide
actgacacggtagacgtccagcctgc

SEQ ID NO: 34 - DNA, 4 bp, Artificial Sequence
  Description of Artificial Sequence Synthetic oligonucleotide
actg

SEQ ID NO: 35 - DNA, 4 bp, Artificial Sequence
  Description of Artificial Sequence Synthetic oligonucleotide
cagt

SEQ ID NO: 36 - DNA, 16 bp, Artificial Sequence
  Description of Artificial Sequence Synthetic oligonucleotide
ctggatcgtatgcgta

SEQ ID NO: 37 - DNA, 7 bp, Artificial Sequence
  Description of Artificial Sequence Synthetic oligonucleotide
gcaggct

SEQ ID NO: 38 - DNA, 16 bp, Artificial Sequence
  Description of Artificial Sequence Synthetic oligonucleotide
acacggtagacgtcca

SEQ ID NO: 39 - DNA, 3 bp, Artificial Sequence
  Description of Artificial Sequence Synthetic oligonucleotide
gtc

SEQ ID NO: 40 - DNA, 16 bp, Artificial Sequence
  Description of Artificial Sequence Synthetic oligonucleotide
tggacgtctaccgtgt

SEQ ID NO: 41 - DNA, 15 bp, Artificial Sequence
  Description of Artificial Sequence Synthetic oligonucleotide
acgcataccatccag

SEQ ID NO: 42 - DNA, 6 bp, Artificial Sequence
  Description of Artificial Sequence Synthetic oligonucleotide
gcctgc

SEQ ID NO: 43 - DNA, 30 bp, Artificial Sequence
  Description of Artificial Sequence Synthetic oligonucleotide
ctgagtacctgattggcttagtcggaagct

SEQ ID NO: 44 - DNA, 30 bp, Artificial Sequence
  Description of Artificial Sequence Synthetic oligonucleotide
acctaggcgtactatcaggtactcagttaa

SEQ ID NO: 45 - DNA, 30 bp, Artificial Sequence
  Description of Artificial Sequence Synthetic oligonucleotide
gaatgccgcttacagtacgcctaggttcga

SEQ ID NO: 46 - DNA, 30 bp, Artificial Sequence
  Description of Artificial Sequence Synthetic oligonucleotide
tccgactaagccagtaagcggcattcctag

SEQ ID NO: 47 - DNA, 4 bp, Artificial Sequence
  Description of Artificial Sequence Synthetic oligonucleotide
agct

SEQ ID NO: 48 - DNA, 4 bp, Artificial Sequence
  Description of Artificial Sequence Synthetic oligonucleotide
ttaa

SEQ ID NO: 49 - DNA, 4 bp, Artificial Sequence
  Description of Artificial Sequence Synthetic oligonucleotide
tcga

SEQ ID NO: 50 - DNA, 4 bp, Artificial Sequence
  Description of Artificial Sequence Synthetic oligonucleotide
ctag

SEQ ID NO: 51 - DNA, 13 bp, Artificial Sequence
  Description of Artificial Sequence Synthetic oligonucleotide
tggcttagtcgga

SEQ ID NO: 52 - DNA, 13 bp, Artificial Sequence
  Description of Artificial Sequence Synthetic oligonucleotide
atcaggtactcag

SEQ ID NO: 53 - DNA, 13 bp, Artificial Sequence
  Description of Artificial Sequence Synthetic oligonucleotide
agtacgcctaggt

SEQ ID NO: 54 - DNA, 13 bp, Artificial Sequence
  Description of Artificial Sequence Synthetic oligonucleotide
gtaagcggcattc

SEQ ID NO: 55 - DNA, 13 bp, Artificial Sequence
  Description of Artificial Sequence Synthetic oligonucleotide
ctgagtacctgat

SEQ ID NO: 56 - DNA, 13 bp, Artificial Sequence
  Description of Artificial Sequence Synthetic oligonucleotide
acctaggcgtact

SEQ ID NO: 57 - DNA, 13 bp, Artificial Sequence
  Description of Artificial Sequence Synthetic oligonucleotide
gaatgccgcttac

SEQ ID NO: 58 - DNA, 13 bp, Artificial Sequence
  Description of Artificial Sequence Synthetic oligonucleotide
tccgactaagcca

SEQ ID NO: 59 - DNA, 30 bp, Artificial Sequence
  Description of Artificial Sequence Synthetic oligonucleotide
ttgctggatccgcatgacattcgccgtaag

SEQ ID NO: 60 - DNA, 30 bp, Artificial Sequence
  Description of Artificial Sequence Synthetic oligonucleotide
cgtttggatccgcatgacattcgccgtaag

SEQ ID NO: 61 - DNA, 30 bp, Artificial Sequence; identical to SEQ ID NO: 4
  Description of Artificial Sequence Synthetic oligonucleotide

SEQ ID NO: 62 - DNA, 26 bp, Artificial Sequence
  Description of Artificial Sequence Synthetic oligonucleotide
tggatccgcatgacattcgccgtaag

SEQ ID NO: 63 - DNA, 30 bp, Artificial Sequence; identical to SEQ ID NO: 8
  Description of Artificial Sequence Synthetic oligonucleotide

SEQ ID NO: 64 - DNA, 30 bp, Artificial Sequence
  Description of Artificial Sequence Synthetic oligonucleotide
gcaacttacggcgaatgaccgaatcagcct

SEQ ID NO: 65 - DNA, 26 bp, Artificial Sequence
  Description of Artificial Sequence Synthetic oligonucleotide
cttacggcgaatgaccgaatcagcct

SEQ ID NO: 66 - DNA, 26 bp, Artificial Sequence
  Description of Artificial Sequence Synthetic oligonucleotide
cttacggcgaatgaccgaatcagcct

SEQ ID NO: 67 - DNA, 30 bp, Artificial Sequence; identical to SEQ ID NO: 13
  Description of Artificial Sequence Synthetic oligonucleotide

SEQ ID NO: 68 - DNA, 30 bp, Artificial Sequence; identical to SEQ ID NO: 14
  Description of Artificial Sequence Synthetic oligonucleotide

SEQ ID NO: 69 - DNA, 30 bp, Artificial Sequence
  Description of Artificial Sequence Synthetic oligonucleotide
aacgaggctgattcggttcatgcggatcca

SEQ ID NO: 70 - DNA, 26 bp, Artificial Sequence
  Description of Artificial Sequence Synthetic oligonucleotide
aggctgattcggttcatgcggatcca

SEQ ID NO: 71 - DNA, 14 bp, Artificial Sequence
  Description of Artificial Sequence Synthetic oligonucleotide
atccttatcaatat

SEQ ID NO: 72 - DNA, 14 bp, Artificial Sequence
  Description of Artificial Sequence Synthetic oligonucleotide
tggtgggttataat

SEQ ID NO: 73 - DNA, 13 bp, Artificial Sequence
  Description of Artificial Sequence Synthetic oligonucleotide
gctgtgaaccaag

SEQ ID NO: 74 - DNA, 14 bp, Artificial Sequence
  Description of Artificial Sequence Synthetic oligonucleotide
cgtctctacctgat

SEQ ID NO: 75 - DNA, 12 bp, Artificial Sequence
  Description of Artificial Sequence Synthetic oligonucleotide
aattaacaataa

SEQ ID NO: 76 - DNA, 36 bp, Artificial Sequence
  Description of Artificial Sequence Synthetic oligonucleotide
ttaacaataatggatccgcatgacattcgccgtaag

SEQ ID NO: 77 - DNA, 39 bp, Artificial Sequence
  Description of Artificial Sequence Synthetic oligonucleotide
aatcactgacatgtggatccgcatgacattcgccgtaag

SEQ ID NO: 78 - DNA, 38 bp, Artificial Sequence
  Description of Artificial Sequence Synthetic oligonucleotide
acgcagtattattggatccgcatgacattcgccgtaag

SEQ ID NO: 79 - DNA, 39 bp, Artificial Sequence
  Description of Artificial Sequence Synthetic oligonucleotide
tactattgcatcttggatccgcatgacattcgccgtaag

SEQ ID NO: 80 - DNA, 41 bp, Artificial Sequence
  Description of Artificial Sequence Synthetic oligonucleotide
ggattattgttaatttggatccgcatgacattcgccgtaag

SEQ ID NO: 81 - DNA, 27 bp, Artificial Sequence
  Description of Artificial Sequence Synthetic oligonucleotide
ggattattgttaaatattgataaggat

SEQ ID NO: 82 - DNA, 27 bp, Artificial Sequence
  Description of Artificial Sequence Synthetic oligonucleotide
catgtcagtgattattataacccacca

SEQ ID NO: 83 - DNA, 25 bp, Artificial Sequence
  Description of Artificial Sequence Synthetic oligonucleotide
ataatactgcgtcttggttcacagc

SEQ ID NO: 84 - DNA, 27 bp, Artificial Sequence
  Description of Artificial Sequence Synthetic oligonucleotide
agatgcaatagtaatcaggtagagacg

SEQ ID NO: 85 - DNA, 36 bp, Artificial Sequence
  Description of Artificial Sequence Synthetic oligonucleotide
cgaccgatgaatagcggtcagatccgtacctactcg

SEQ ID NO: 86 - DNA, 36 bp, Artificial Sequence
  Description of Artificial Sequence Synthetic oligonucleotide
cgagtaggtacggatctgcgtattgcgaacgactcg

SEQ ID NO: 87 - DNA, 36 bp, Artificial Sequence
  Description of Artificial Sequence Synthetic oligonucleotide
cgagtcgttcgcaatacggctgtacgtatggtctcg

SEQ ID NO: 88 - DNA, 36 bp, Artificial Sequence
  Description of Artificial Sequence Synthetic oligonucleotide
cgagaccatacgtacagcaccgctattcatcggtcg

SEQ ID NO: 89 - DNA, 26 bp, Artificial Sequence
  Description of Artificial Sequence Synthetic oligonucleotide
tggatccgcatgacattcgccgtaag

SEQ ID NO: 90 - DNA, 30 bp, Artificial Sequence
  Description of Artificial Sequence Synthetic oligonucleotide
actcaggctgattcggttcatgcggatcca

SEQ ID NO: 91 - DNA, 26 bp, Artificial Sequence
  Description of Artificial Sequence Synthetic oligonucleotide
cttacggcgaatgaccgaatcagcct

SEQ ID NO: 92 - DNA, 30 bp, Artificial Sequence
  Description of Artificial Sequence Synthetic oligonucleotide
gagttggatccgcatgacattcgccgtaag

SEQ ID NO: 93 - DNA, 30 bp, Artificial Sequence; identical to SEQ ID NO: 13
  Description of Artificial Sequence Synthetic oligonucleotide

SEQ ID NO: 94 - DNA, 30 bp, Artificial Sequence; identical to SEQ ID NO: 64
  Description of Artificial Sequence Synthetic oligonucleotide

SEQ ID NO: 95 - DNA, 26 bp, Artificial Sequence
  Description of Artificial Sequence Synthetic oligonucleotide
aggctgattcggttcatgcggatcca

SEQ ID NO: 96 - DNA, 26 bp, Artificial Sequence
  Description of Artificial Sequence Synthetic oligonucleotide
cttacggcgaatgaccgaatcagcct

SEQ ID NO: 97 - DNA, 90 bp, Artificial Sequence
  Description of Artificial Sequence Synthetic oligonucleotide
cttacggcgaatgaccgaatcagcctgagttggatccgcatgacattcgccgtaagttgcttgcaggctgattcggttcatgcggatcca

SEQ ID NO: 98 - DNA, 86 bp, Artificial Sequence
  Description of Artificial Sequence Synthetic oligonucleotide
aggctgattcggttcatgcggatccagcataggctgattcggttcatgcggatccaactcaggctgattcggttcatgcggatcca

SEQ ID NO: 99 - DNA, 90 bp, Artificial Sequence
  Description of Artificial Sequence Synthetic oligonucleotide
cttacggcgaatgaccgaatcagcctgcaagcaacttacggcgaatgaccgaatcagcctatgctggatccgcatgacattcgccgtaag

(19) United States
(12) Patent Application Publication
(10) Pub. No.: US 2007/0048759 A1
(43) Pub. Date: Mar. 1, 2007

(54) DETECTION OF TARGET MOLECULES WITH LABELED NUCLEIC ACID DETECTION MOLECULES

(76) Inventors: Dan Luo, Ithaca, NY (US); Yougen LI, Pasadena, CA (US)

Correspondence Address: WILSON SONSINI GOODRICH & ROSATI, 650 PAGE MILL ROAD, PALO ALTO, CA, 94304-1050, US

(21) Appl. No.: 11/423,633

(22) Filed: Jun. 12, 2006

Related U.S. Application Data
(60) Provisional application No. 60/689,285, filed on Jun. 10, 2005.
(60) Provisional application No. 60/783,422, filed on Mar. 17, 2006.
(60) Provisional application No. 60/783,426, filed on Mar. 17, 2006.
(60) Provisional application No. 60/745,383, filed on Apr. 21, 2006.
(60) Provisional application No. 60/756,453, filed on Jan. 5, 2006.

(51) Int. Cl. C12Q 1/68; C07H 21/04

(52) U.S. Cl. 435/6; 536/24.3

(57) ABSTRACT

The invention is directed to a detection molecule for detection of a target molecule. The detection molecule includes a probe specific to the target molecule. One or more multimer nucleic acid molecules are connected to the probe, whereby the multimer is also coupled to at least one detectable label. The detection molecules are utilized in a method to detect the presence of one or more target molecules in a sample.

### CROSS-REFERENCE TO RELATED APPLICATIONS

The present application claims priority to provisional application Ser. No. 60/689,285, filed Jun. 10, 2005, provisional application Ser. No. 60/745,383, filed Apr. 21, 2006 and 60/783,426, filed Mar. 17, 2006, the disclosures of which are hereby incorporated by reference in their entirety. Applicants claim the benefits of this application under 35 U.S.C. §119 (e) and/or §35 U.S.C, 120.

### GOVERNMENT BACKED WORK

The invention was made, at least in part, with the support of a grant from the Government of the United States of America (grant ECS-9876771 from the National Science Foundation). The U.S. Government may have certain rights to the invention.

### FIELD OF THE INVENTION

The invention relates to the detection of target molecules in samples with labeled nucleic acid detection molecules.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

A key aim of biotechnology and nanotechnology is the construction of new biomaterials, including individual geometrical objects, nanomechanical devices, and extended constructions that permit the fabrication of intricate structures of materials to serve many practical purposes (Feynman et al., Miniaturization 282-296 (1961); Drexler, Proc. Nat. Acad. Sci. 78:5275-5278 (1981); Robinson et al., Prot. Eng 1 295-300(1987); Seeman, DNA & Cell Biol. 10:475-486 (1991); Seeman, Nanotechnol. 2:149-159 (1991)). Molecules of biological systems, for example, nucleic acids, have the potential to serve as building blocks for these constructions due to their self and programmable-assembly capabilities.

DNA molecules possess a distinct set of mechanical, physical, and chemical properties. From a mechanical point of view, DNA molecules can be rigid (e.g., when the molecules are less than 50 nm, the persistent length of double stranded DNA (Bouchiat, C. et al., Biophys. J. 76:409-13 (1999); Tinland et al., Macromolec. 30:5763-5765 (1997); Toth et al., Biochem. 37:8173-9 (1998)), or flexible. Physically, DNA is small, with a width of about 2 nanometers and a length of about 0.34 nanometers per basepair (for B-DNA). In nature, DNA can be found in, either linear or circular shapes. Chemically, DNA is generally stable, non-toxic, water soluble, and is commercially available in large quantities and in high purity. Moreover, DNA molecules are easily and highly manipulable by various well-known enzymes such as restriction enzymes and ligases. Also, under proper conditions,, DNA molecules will self-assemble with complimentary strands of nucleic acids e.g., DNA, RNA, or Peptide Nucleic Acid, (PNA) or with proteins. Furthermore, DNA molecules can be amplified exponentially and ligated specifically. Thus, DNA is an excellent candidate for constricting nano-materials.

The concept of using DNA molecules for non-genetic application has only recently emerged as two new fields of research DNA-computation, such as using DNA in algorithms for solving combinatorial problems (Adleman, Science 266:1021-4 (1994), Guarnieri et al., Science 273:220-3 (1996); Ouyang et al., Science 278:446-9 (19970); Sakamoto et al., Science 288:1223-6 (2000) Benenson et al., Nature 414:430-4 (2001)), and DNA-nanotechnology, such as using DNA molecules for nano-scaled frameworks and scaffolds (Niemeyer, Appl. Phys. Matls. Sci. & Proc. 68:-119-124 (1999); Seeman, Ann. Rev. Biophy. & Biomolec. Struct. 27:225-248 (1998)). However, the design and production of DNA-based materials is still problematic (Mao et al., Nature 397:144-146 (1999); Seeman et al., Proc Natl Acad Sci USA 99:64501-6455 (2002); Yan et al., Nature 415:62-5 (2002); Mirkin et al., Nature 382:607-9 (199); Watson et al., J. Am. Chem. Soc. 123:5592-3 (2001)). For example, previously reported nucleic acid structures were quite polydispersed with flexible arms and self-ligated circular and non-circular by-products (Ma et al., Nucl. Acids Res. 14:9745-53 (1986); Wang et al., J. Amer. Chem. Soc. 120:8281-8282 (1998); Nilsen et al., J. Theor. Biol. 187:273-84 (1997)), which severely limited their utility in constructing DNA materials. The yield and purity of those structures were also unknown.

Alderman first solved an instance of the directed Hamiltonian path problem using DNA molecules and reactions (Adleman, Science 266-1021-4 (1994)). Since then, DNA has been used as algorithms for solving combinatorial problems (Guarnieri et at. Science 273:220-3 (1996); Ouyang et al., Science 278:446-9 (1997); Sakamoto et al., Science 288:1223-6 (2000); Benenson et al., Nature 414:430-4 (2001)) and logical computation (Mao et al., Nature 407:493-6 (2001)). However, in all of these applications, the shapes of DNA molecules have not been altered; they are still in linear form as the hairpin form was employed, which is also a linear form.

The field of DNA nanotechnology was pioneered by Seeman (Seeman, J. Biomol. Struct. Dyn. 8:573-81 (1990); Seeman, Accnts. Chem. Res. 30:357-363 (1997); Seeman, Trends Biotech. 17:437-443 (1999)). Using rigid “crossover” DNA as building blocks motifs were constructed (Seeman et al., Biophys. J. 78:308a-308a (2000); Sha et at., Chem. & Biol. 7:743-751 (2000); LaBean et al., J. Amer. Chem. Soc. 122:1848-1860 (2000); Yang et al., J. Amer. Chem. Soc. 120:9779-9786 (1998); Mao et al., Nature 386:137-138 (1997)). A DNA mechanical device was also reported (Mao et al., J. Amer. Chem. Soc. 121:5437-5443 (1999); Yan et al., Nature 415:62-5 (2002)). However, the building blocks and motifs employed so far are isotropic multivalent, possibly useful for growing nano-scaled arrays and scaffolds (Winfree et al., Nature 394:539-4 (1998); Niemeyer, Appl. Phys. Matl. Sci. & Proc. 68:119-124 (1999); Seeman, Ann. Rev. Biophys. & Biomolec. Struct. 27:225-248 (1998)), but not suitable for controlled growth, such as dendrimers, or in creating a large quantity of monodispersed new materials, which are important to realize nucleic acid-based materials.

Other schemes of nano-construction using linear DNA molecules were also reported, including a biotin-avidin-based DNA netork (Luo, “Novel Crosslinking Technologies to Assess Protein-DNA Binding and DNA-DNA Complexes for Gene Delivery and Expression” (Dissertation) and dendrimer-like DNA (Li et al., “Controlled Assembly of Dendrimer-like DNA,” Nature Materials 3:138-42 (2004)).

Molecular, Cellular, and Developmental Biology Program, The Ohio State University (1997)), nanocrystals (Alivisatos et al., Nature 382:609-11 (1996)), DNA-protein nanocomplexes (Niemeyer et al., Angewandte Chemi-Inter. Ed. 37:2265-2268 (1998)), a DNA-fueled molecular machine (Yurke et al., Nature 406:605-8 (2000)), DNA-block copolymer conjugates (Watson et al., J. Am. Chem. Soc. 123:5592-3 (2001)), DNA-silver-wire (Braun et al., Nature 391:775-8 (1998)), and DNA-mediated supramolecular structures (Taton et al., J. Amer. Chem. Soc. 122:6305-6306 (2000). In addition, Mirkin has reported DNA sensing via gold nanoparticles (Elghanian et al., Science 277:1078-81 (1997)) and DNA patterning via dip-pen nanolithography (Demers et al., Science 296:1836-8 (2002)), although such patterning is not suitable for large scale production.

Recently, Mirkin's group reported a DNA array-based detection method that utilized microelectrodes (Park et al., Science 295:1503-1506 (2002)). DNA-based lithography was recently reported by Braun's group where linear and very long DNA molecules were reported to serve as masks and RecA proteins served as resists (Keren et al., Science 297:72-5 (2002)). Recently, a small chemical, tris-linker was reportedly reacted with 5′-hydrazide-modified oligonucleotides in the presence of the 3′-tis-oligonucleotidyl template to allegedly create tri-valent, Y-shape DNA molecules (Eckardt et al., Nature 420:286 (2002)). However, all of these examples involved linear DNA.

Rapid, multiplexed, sensitive, and specific molecular detection is of great demand in gene profiling, drug screening, clinical diagnostics, and environmental analysis (Han et al., “Quantum-Dot-Tagged Microbeads for Multiplexed Optical Coding of Biomolecules,” Nature Biotech. 19:631-635 (2001); Fulton et al., “Advanced Multiplexed Analysis with the FlowMetrix(TM) System,” Clin. Chem. 43-1749-1756 (1997); and Steemers et al., “Screening Unlabeled DNA Targets with Randomly Ordered Fiber-Optic Gene Arrays,” Nature Biotech. 18-91-94 (2000)). One of the major challenges in multiplexed analysis is to identify each reaction with a code (Braeckmans et al., “Encoding Microcarriers Present and Future Technologies,” Nature Rev. Drug Discov. 1:447-456(2002)).

Two encoding strategies are currently used positional encoding in which every potential reaction is pre-assigned a particular position such as on a solid-phase DNA microarray (Duggan et al., “Expression Profiling Using cDNA Microarrays,” Nature Genet. 21:10-14 (1999); DeRisi et al., “Use of a cDNA Microarray to Analyse Gene Expression Patterns in Human Cancer,” Nature Genet. 14:457-460 (1996); Schena et al., “Quantitative Monitor of Gene Expression Pattern with a Complementary DNA Microarray,” Science 270:467-470 (1995); and Cheung et al., “Making and Reading Microarrays,” Nature Genet. 21:15-19 (1999)), and reaction encoding, where every possible reaction is uniquely tagged with a code that is mostly optical or particle based (Braeckmans et al., “Encoding Microcarriers Present and Future Technologies,” Nature Rev. Drug Discov. 1:447-456 (2002); Cunin et at., “Biomolecular Screening with Encoded Porous-silicon Photonic Crystals,” Nature Matl. 1:39-41 (2002); Wang et at., “Encoded Beads for Electrochemical Identification,” Analyt. Chem. 75:4667-4671 (2003); Chan et al., “Luminescent Quantum Dots for Multiplexed Biological Detection and Imaging,” Curr. Op. Biotech. 13:40-46 (2002); Ried et al., “Simultaneous Visualization of 7 Different DNA Probes by In situ Hybridization Using Combinatorial Fluorescence and Digital Imaging Microscopy,” Proc. Nat'l. Acad. Sci. USA 89:1388-1392 (1992); and Nicewarner-Pena et al., “Submicrometer Metallic Barcodes,” Science 294:137-141 (2001)).

Micrometer size, polydispersity the complex fabrication, and non-biocompatibility of current codes continuously limit their usability (Han et al., “Quantum-Dot-Tagged Microbeads for Multiplexed Optical Coding of Biomolecules,” Nature Biotech. 19:631-635 (2001); Braeckmans et. al., “Encoding Microcarriers Present and Future Technologies,” Nature Rev. Drug Discov. 1447-456 (2002); and Nicewarner-Pena et al., “Submicrometer Metallic Barcodes” Science 294:137-141 (2001)).

Therefore the invention is directed to satisfying the need for dendrimer-like DNA based, fluorescence-intensity-coded nanobarcodes, which have both built-in codes and molecular probes for molecular sensing.

### SUMMARY OF THE INVENTION

In one aspect of the invention, the successful synthesis and application of dendrimer like nucleic acid molecules (DL-NAM) reveals two novel concepts 1) multiplexed detection can be achieved by detecting different fluorescent intensity ratios instead of different fluorescent colors, and 2) DL-NAM can be used as both structure scaffoldings and functional probes. In certain embodiments, the compositions and methods are directed to precisely controlled fluorescence intensity ratios at the individual molecular level, which are achieved with anisotropic, multivalent carriers, such as DL-NAM. In addition, this ability to precisely manipulate specific nanobarcodes onto individual DNA nano structure has enabled the achievement of single molecular detection. Furthermore, the nucleic acid scaffold and the detectable labels make the nanobarcodes biocompatible and thus can be applied in vivo. The DNA scaffold also makes nanobarcodes highly modifiable due to the existence of a myriad of DNA modification enzymes that are conventional in the art.

Another aspect is directed to a detection molecule for detection of a target molecules The detection molecule comprises a probe specific to the target molecule, one or more multimer nucleic acid and one or more labels. Furthermore, one or more multimer nucleic acid molecule is linked to the probe, which multimers include trimers and tetramer shaped molecules. A trimer comprises a first, a second, and a third polynucleotide, where a least a portion of the first polynucleotide is complementary to at least a portion of the second polynucleotide, at least a portion of the first polynucleotide is complementary to at least a portion of the third polynucleotide, and at least a portion of the second polynucleotide is complementary to at least a portion of the third polynucleotide. The polynucleotide are associated together to form a trimer. In addition, one or more label molecules are coupled to each trimer. Furthermore, the trimer can be Y-shape or T-shape as described herein. In one embodiment, a detection molecule is comprised of two trimers ligated together to form a dumbbell-shape.

A tetramer comprises a fourth polynucleotide, in addition to a first, second and third polynucleotide, where at least a portion of the first polynucleotide is complementary to the second and fourth polynucleotide,, where at least a portion of the second polynucleotide is complementary to the first and third polynucleotide, where at least a portion of the third polynucleotide is complementary to the second and fourth polynucleotide and where the polynucleotides are associated together to form a tetramer. In addition one or more label molecules are coupled to each tetramer. In one embodiment the tetramer is X-shaped. In another embodiment, the tetramer molecule is dumbbell-shaped.

A further aspect of the invention is directed to a method of detecting a target molecule, if present in a sample, where detection is facilitated by utilization of the detection molecules. The method for detection includes providing a detection molecule which comprises a probe specific to the target molecule, which probe is connected to one or more multimer nucleic acid molecules. Multimers include trimer or tetramer shapes comprising polynucleotides as described herein. Furthermore, one or more label molecule providing a detectable signal is linked to at least one trimer or one tetramer.

In one embodiment, a sample is contacted with the detection molecule under conditions effective to permit target molecules to specifically bind to the probe of the detection molecule, any specific binding of target molecules to the probe of the detection molecule is detected via the detectable one or more label molecules, thereby detecting the presence of target molecule in the sample.

The detection molecules (also referred to as, barcodes or nanobarcodes, DL-NAM) will find a wide range of applications in both in vitro and in vivo, especially intracellular applications (e.g. intracellular and/or in situ multiplexed detections). Unlike solid-phase based DNA microarrays where cells and tissues must be lysed first and nucleic acids are then extracted before adding them onto a microarray destroying all community (in situ) information, the reported nanobarcodes, are solution-based, nanoscale, “soft” arrays that can be applied directly onto tissues or cells, making in situ multiplexed detection possible. The use of common and commercially available fluorophores does not require special equipment for detection, effectively expanding die power of traditional microscopy. For example, a microscope with only two common color filters can now be used to simultaneously image at least 5 different targets labeled with only two colors as reported here. In addition, this technique could also substitute both isotope and fluorochrome labelling for blotting-based, simultaneous, multiplexed detection without resorting to multiple runs or repeating probe stripping, as practiced at present. Furthermore, detection molecules allow multiplexed flow cytometry with only two colors possible, resulting in detection of target molecules or analytes that is both fast and sensitive. In other embodiments, the labels can be any molecule providing a detectable signal, as further described herein, and such molecules include enzymes, enzyme substrates, proteins, peptides and quantum dots.

In some embodiments, the detection molecules are utilized in fluorescence microscopy, dot blotting, and flow cytometry. As a result the following, is apparent 1) nucleic acids, especially DL-NAM, has been employed as both the structural scaffoldings and functional probes; 2) a paradigm shift has been validated for multiplexed molecular sensing that relies on the detection of precise fluorescent color ratios instead of the detection of single colors; and 3) a nucleic acid-based, multiplexed sensing platform nanotechnology has been realized which can be applied in almost any fluorescence-based detection system. This technology can be widely employed in a myriad of applications, from in situ hybridization to genomic research, from clinical diagnosis to drug discovery, and from environmental monitoring to anti-bioterrorism (e.g., detection of biowarfare/bioterror biologicals, such as virus, bacteria, etc.).

### BRIEF DESCRIPTION OF THE FIGURES

FIG. 1 is a schematic drawing of DNA molecular assembly. FIG. 1A depicts the assembly of Y-DNA. The oligonucleotides were annealed together to form one Y-shape DNA, a basic building block for dendrimer-like DNA (Y₀ₐ+Y_{0b}+Y_{0c}→Y₀; Y₁ₐ+Y_{1b}+Y_{1c}→Y₁; Y₂ₐ+Y_{2b}+Y_{2c}→Y₂; Y₃ₐ+Y_{3b}+Y_{3c}→Y₄ₐ+Y_{4b}+Y_{4c}→Y₄). FIG. 1B depicts the assembly of first generation dendrimer-like DNA (G₁). The core Y₀-DNA was ligated with three “Y₁”s, all with specifically designed sticky ends. The ligation was unidirectional. FIG. 1C depicts the assembly of second generation dendrimer-like DNA (G₂). G₁ DNA was ligated with six Y₂-DNAs. FIG. 1D depicts the assembly of third generation dendrimer-like DNA (G₃) and G₄.

FIG. 2 depicts divergent and convergent synthesis of a nucleic acid assembly

FIG. 3 depicts dendrimer-like DNA.

FIG. 4 is a schematic drawings of G₂ DL-NAM (left) and other higher generation DL-NAM (right).

FIGS. 5A-D depict the synthesis of barcodes. FIG. 5A shows a schematic illustration of the synthesis of a Y-DNA based barcode building block. Three starting oligonucleotide components were partially complementary (SEQ ID NOs: 59, 65 and 70 (top to bottom, respectively)). One oligonucleotide possessed a sticky end, another one was labeled with a fluorescent dye, and the third one was labeled with a fluorescent dye or a probe depending on the experimental design. FIG. 5B shows a schematic illustration of constructing a DL-NAM-based nanobarcode. The barcode building blocks were covalently linked with each other through complementary sticky ligations. FIGS. 5C shows a schematic illustration of barcode decoding. The nanobarcodes 4G1R, 2G1R, 1G1R, 1G2R, and 1G4R were decoded based on the ratio of fluorescence intensity (where G=green label and R=Red label, and a numeral before either G or R indicates the number of labels present on any particular multimer nucleic acid molecule). A molecular recognition element, a probe, was also attached to each barcode (i.e., probes 1-5 are depicted) where the probes can be specific to a particular target molecule (e.g., each probe is specific to a different target molecule). The resultant nanobarcodes were comprised of not only coding capacity, but also molecular sensing ability. With a pre-assigned code library, the barcodes could be used for molecular detection of a plurality of different target molecules. As shown in FIG. 5D, the real color of barcodes in an agarose gel illuminated with a strong UV light. Lanes 1 and 7 are Alexa Fluor 488 labeled starting oligonucleotide component and Bodipy 630/650 labeled starting oligonucleotide component, respectively. Lanes 2, 3, 4, 5, 6 are barcodes 4G1R, 2G1R, 1G1R, 1G2R, and 1G4R respectively.

FIGS. 6A-C shows the microbead-based DNA detection using fluorescence microscopy. FIG. 6A is a schematic drawing of a barcode signal amplification strategy achieved from polystyrene microbead based, sandwiched DNA hybridizations. Briefly, biotin-labeled capture probes were attached to avidin functionalized polystyrene microbeads. Each batch of microbeads consisted of only one type of capture probe before pooling them together. DNA targets (i.e. control or unknown samples) were then captured by specific microbeads first. Each report probe which was linked to a particular nanobarcode, was designed to be complementary to another part of a specific target DNA and thus was able to be hybridized onto a specific microbead. Since each microbead bound a large amount of sandwiched complexes (i.e.. capture probes/target DNA/report probes/nanobarcodes), fluorescence signals were amplified. FIG. 6B shows the merged fluorescent colors (pseudocolors) of barcodes from individual microbeads. FIG. 6C shows the multiple target detections (a total of 5 targets) were achieved via a two-colored fluorescence microscope using DNA barcodes and microbeads. Scale bars are all 5 μm,

FIGS. 7A-B depict a DNA blotting assay with barcodes. FIG. 7A shows a schematic drawing of a dot blotting detection of multiple DNA targets with barcodes. Target DNA molecules were manually blotted onto a nylon-membrane. After pre-hybridization and blocking, a library of barcode mixture was loaded onto the membrane. Through specific hybridizations with report probes, which were functionalized with barcodes, target DNA molecules were detected using a fluorescence reader, scanner, or microscope. As shown in FIG. 7B, multiple pathogens (four total) were detected simultaneously using nanobarcodes. The control 1 was a 27-mer ssDNA with unrelated sequences and the control 2 was a plasmid DNA, pVAX1/lacZ. Scale bar: 1 mm.

FIGS. 8A-B show the evaluation of barcodes with agarose gel electrophoresis. FIG. 8A shows the evaluation of DNA barcodes with 3% agarose gel electrophoresis. Lane 1 is a starting oligonucleotide component (30-mer), and lanes 2, 3 4, 5, 6 are barcodes 4G1R, 2G1R, 1G1R, 1G2R, 1G4R, respectively. FIG. 8B shows the evaluation of denatured DNA barcodes with 3% agarose gel electrophoresis. Lanes 1 and 2 are starting oligonucleotide components (30-mer and 42-mer) as molecular markers. Lanes 3, 4, 5, 6, 7 are denatured 4G1R, 2G1R, 1G1R, 1G2R, 1G4R, respectively. Schematic drawings of denatured barcodes are shown in FIGS. 9A-D.

FIGS. 9A-D show schematic drawings of barcode denaturation (without showing fluorescence dyes). FIG. 9A is the barcode 4G1R or 1G4R; FIG. 9C is the barcode 1G1R; FIG. 9C is the barcode 2G1R; and FIG. 9D is the barcode 1G2R. Gel electrophoresis of denatured products are shown in FIG. 8.

FIG. 10 shows the DNA barcode quantitative decoding based on microbead populations.

FIGS. 11A-B show the multiplexed DNA detection using flow cytometry. FIG. 11A shows a two-color flow plot of microbeads attached with the barcode 2G1R as a control for standards (a calibration control). FL1H is a green channel and FL4H is a red channel. FIG. 11B shows the simultaneous detection of three pathogens using nanobarcodes. Unrelated DNA sequences were not detected (background).

FIGS. 12A-B are schematic drawings of an X-shaped nucleic acid molecular assembly. FIG. 12A depicts the assembly of X-shaped nucleic acid. Four oligonucleotides were annealed together to form one X-shaped nucleic acid, a basic building block for dendrimer-like nucleic acid. FIG. 12B shows the assembly of a plurality of X-shaped nucleic acid molecules into a dendrimer structure.

FIGS. 13A-B are schematic drawings of an T-shaped nucleic acid molecular assembly. FIG. 13A depicts the assembly of T-shaped nucleic acid. Three oligonucleotides were annealed together to form one T-shaped nucleic acid, a basic building block for dendrimer-like nucleic acid. FIG. 13B shows the assembly of a plurality of T-shaped nucleic acid molecules into a dendrimer structure.

FIGS. 14A-B show the nucleotide sequences of the polynucleotides (SEQ ID NOS: 44, 45, 46 and 43, respectively) used to make the X-shaped tetramer individually (FIG. 14A) and together (FIG. 14B).

FIGS. 15A-B show the nucleotide sequences of the polynucleotides (SEQ ID NOS: 31-33) used to make the T-shaped trimer individually (FIG. 15A) and together (FIG. 15B).

FIG. 16 shows X-shaped nucleic acid molecules and Y-shaped nucleic acid molecules joined together.

FIG. 17 shows X-shaped nucleic acid molecules and T-shaped nucleic acid molecules joined together.

FIG. 18 shows T-shaped nucleic acid molecules and Y-shaped nucleic acid molecules joined together.

FIG. 19 shows X-shaped nucleic acid molecules, Y-shaped nucleic acid molecules, and T-shaped nucleic acid molecules joined together.

### DETAILED DESCRIPTION OF THE INVENTION

Certain aspects of the invention are directed to compositions and methods that provide the first successful multiplexed detection system using -nucleic acid dendrimers to precisely control fluorescence intensity ratios at the individual molecule level. The detection molecules comprise nucleic acid multimers, probes and labels, which are biocompatible and thus can be applied in vivo. In certain embodiments, the detection molecules are comprised of a plurality of multimers that form a nucleic acid scaffold, which nucleic acids are also highly modifiable. Nucleic acids are manipulated functional elements added—due to the existence of a myriad of enzymatic modifications that are conventional in the art with respect to nucleic acids (e.g., Luo, D., The Road From Biology to Materials, Materials Today 2003; Vol. 6: 38-43) A nonexclusive list of such enzymes includes T4 DNA ligase, E. coli POL I, Taq polymerase, reverse transcriptase, terminal transferase, T4 DNA ligase, E. coli DNA ligase, T4 RNA ligase, endonucleases such as λ endonuclease, exonuclease, ribonuclease H, mung bean or micrococcal nuclease, DNases, restriction enzymes, kinases such as T4 kinase, and methylases.

In one aspect of the invention, one or more frictional elements can be easily introduced either before (e.g. the molecular recognition elements can be linked to the oligonucleotide components) or after barcode synthesis. In another embodiment a detection molecule (i.e., barcode) is of nano-scale size, as distinguished to micro-sized structures (Braeckmans et al., “Encoding Microcarriers Present and Future Technologies,” Nature Rev. Drug Dis. 1:447-456 (2002); Cunin et al., “Biomolecular Screening with Encoded Porous-silicon Photonic Crystals,” Nature Mat'l. 1:39-41 (2002); Wang et al., “Encoded Beads for Electrochemical Identification,” Anal. Chem. 75:4667-4671 (2003); Chan et al., “Luminescent Quantum Dots for Multiplexed Biological Detection and Imaging,” Curr. Op. Biotech. 13:40-46 (2002); and Nicewarner-Pena et al., “Submicrometer Metallic. Barcodes,” Science 294:137-141( (2001), which are hereby incorporated by reference in their entirety).

Another aspect of the invention is directed to a detection molecule for detection of a target molecule. The detection molecule includes a probe specific to a particular target molecule or analyte. In one embodiment the probe is linked to a single multimer. In other embodiments, the probe is linked to two or more multimers.

The multimer molecules may be trimer or tetramer shaped. Furthermore, one or more multimer molecules may be coupled to at least one label molecule In some embodiments, the multimer molecule is coupled to a plurality of label molecules. In a further embodiment, the plurality of label molecules includes the same or different molecules, where “different” constitutes a differentially observable signal. In some embodiments, a detection molecule is linked to a quantity of 1, 2, 3, 4, 5, 6, 7, 8, 9, 10, 11, 12, 13, 14, 15, 16, 17, 18, 19 or 20 detection labels. In another embodiment, the detection molecule is linked to 1, 2, 3, 4, 5, 6, 7, 8, 9, or 10 different types of labels. In yet further embodiments, a detection molecule comprises a quantity (i.e., number) of 1, 2, 3, 4, 5, 6, 7, 8, 9, 11, 11, 12, 13, 14, 15, 16, 17 18, 19 or 20 and/or 1 2, 3, 4, 5, 6, 7, 8, 9, or 10 different types of detection labels.

As used herein, the term “multimer” includes a nucleic acid molecule that is a trimer(s) or a tetramer(s), or a combination of trimer(s) and tetramer(s), which terms “trimer” and “tetramer” are further described herein.

Each trimer comprises a first, a second, and a third polynucleotide, and each tetramer comprising a first, second, third and fourth polynucleotide. The trimer molecule can be Y- or T-shape(d), wherein at least a portion of the first polynucleotide is complementary to at least a portion of the second polynucleotide, at least a portion of the first polynucleotide is complimentary to at least a portion of the third polynucleotide, and at least a portion of the second polynucleotide is complementary to at least a port-ion of the third polynucleotide. The first, second, and third polynucleotides are associated together to form a trimer. One or more label molecules are coupled to each trimer.

In one aspect of the invention, the T-shape trimer molecules at least a portion of the first polynucleotide is complementary to a portion of the second polynucleotide, where at least a portion of hie first polynucleotide is complementary to the at least a portion of the third polynucleotide, wherein at least a portion of the second polynucleotide is complementary to at least a portion of the third polynucleotide, and where he third polynucleotide is configured so that the -portion of it that is complementary to the second polynucleotide are essentially co-linear.

In another aspect of the invention, the tetramer molecules of the invention comprise a first, second, third and fourth polynucleotide, where at least a portion of the first polynucleotide is complementary to at least a portion of the second and fourth polynucleotide, at least a portion of the second polynucleotide is complementary to at least a portion of the first and third polynucleotide and at least a portion of the third polynucleotide is complementary to at least a portion of the second and fourth polynucleotide, and at least a portion of the four polynucleotide is complementary to at least a portion of the first and third polynucleotide, In one embodiment, the first, second, third and fourth polynucleotides are associated together to form a tetramer. One or more label molecules may be coupled to each tetramer. In one embodiment, the tetramer is X-shape(d). In another embodiment, the tetramer is dumbbell-shape(d).

In some aspects of the invention, the trimer has three branches. In one embodiment, at least one of the polynucleotides includes a sticky end. In another embodiment, a first region of the first, second and third polynucleotides includes a sticky end. In one embodiment, the polynucleotides are RNA, RNA or DNA, or RNA and DNA, in any combination thereof. In another embodiment, the first, second, and third polynucleotides are DNA.

In another aspect of the invention, the tetramer has four branches. In one embodiment, at least one of the polynucleotides includes a sticky end. In another embodiment the first region of the first, second third and fourth polynucleotide includes a sticky end. In yet further embodiments, the first, second, third and fourth polynucleotides are DNA.

Other aspects of the invention are directed to the polynucleotides that may be incorporated into either the trimer or tetramer nucleic acid molecules of the invention. Non-limiting examples of such other molecules include: DNA, RNA, PNA (peptide nucleic acid), TNA (threose nucleic acids), and other polymers that are able to complex with nucleic acids in a sequence specific manner.

Further aspects of the invention are directed to assembly of multimer nucleic acid molecules, where trimers, tetramers or combinations of both are utilized to assemble a plurality of multimers. In one embodiment, at least two trimers can be associated together. In some embodiments, the trimers are ligated together. In additional embodiments, the assembly forms a honeycomb structure. In yet other embodiments, the assembly forms a dendrimer structure.

In another embodiment, at least two of the tetramers can be associated together. In some embodiments, the tetramers are ligated together. In additional embodiments, the assembly forms a networked structure of tetramer molecules. In yet other embodiments, the assembly forms a dendrimer structure.

In yet another embodiment, a mixture of trimers and tetramers are associated together. In one embodiment, the nucleic acid assembly forms by associating at least two trimers or two tetramers together. In some embodiments, the association step includes ligating at least two trimers together or ligating at least two tetramers together.

In some embodiment, one or more tetramers are ligated to trimers, as well as to one or more tetramers. Furthermore, said trimers may be also ligated to other trimers, as well as to said tetramers, In additional embodiments the assembly forms a three-dimensional dendrimer structure. In one embodiment, dendrimers formed by the multimers are monodisperse or nearly monodisperse.

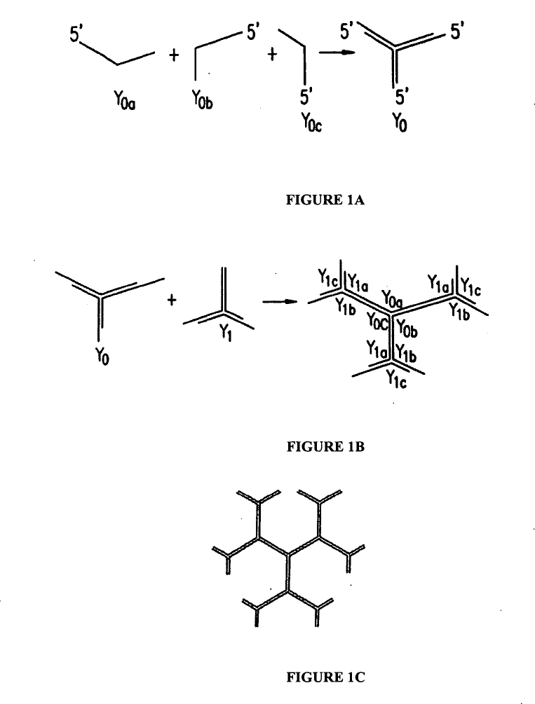

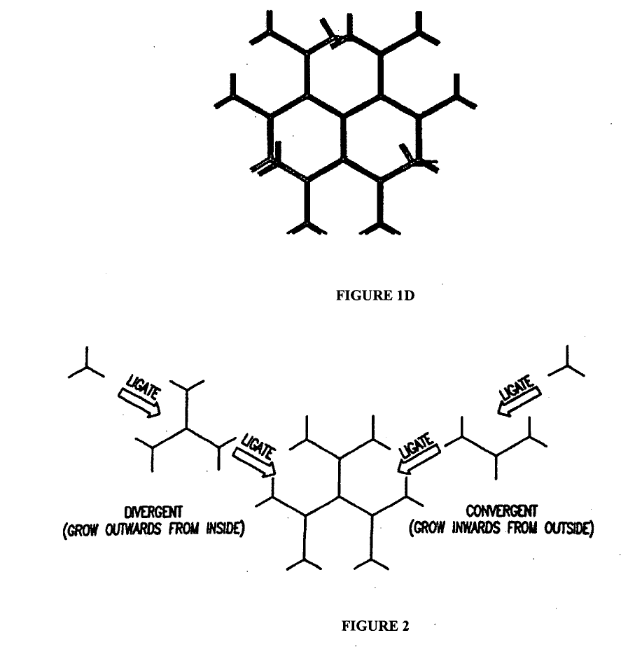

As shown in FIG. 1A, in one embodiment the method of making the trimer includes the following steps: combining a first, a second, and a third polynucleotide in a solution, where at least a portion of the first polynucleotide, is complementary to at least a portion of the second polynucleotide, where at least a portion of the first polynucleotide is complementary to at least a portion of the third polynucleotide, and where at least a portion of the second polynucleotide is complementary to at least a portion of the third polynucleotide. The solution is maintained at conditions effective for the first, second, and third polynucleotides to associate together to form a trimer. Similar procedures are used to form T-shaped, nucleic acid molecules, as shown in FIG. 13A.

In some embodiments for trimer formation, the combining step includes the steps of combining the first polynucleotide with the second polynucleotide in a solution to form a mixture, amid subsequently combining the third polynucleotide with the first and second polynucleotides in the solution. In some embodiments, each polynucleotide includes a first region, a second region located 3′ to the first region, and a third region located 3′ to the second region, wherein the second region of the first polynucleotide includes a region complementary to the third region of the third polynucleotide, die third region of the first polynucleotide includes a region complementary to the second region of the second polynucleotide, the third region of the second polynucleotide includes a region complementary to the second region of the third polynucleotide.

As is described herein, a “trimer” is a structure formed by the association of three polynucleotides (see FIG. 1A or FIG. 13A). The terms “trimer” and “Y-DNA” or “T-DNA” are used interchangeably herein. The trimer is a generally Y-shaped or T-shaped structure having three branches. A “branch” is a structure formed by the association, for example, the hybridization, of portions of two polynucleotides.

In some aspects of tie invention each branch may have a sticky end. A sticky end is a single-stranded overhang portion of one of the polynucleotides forming the branch of a trimer or tetramer molecule. In some embodiments, the sticky ends can be 4, 5, 6, 7, 8, 9 or 10 nucleotides. The sticky end in some embodiments is a four nucleotide sticky end. Sticky ends are designed so that the sticky ends of polynucleotides in different trimers will hybridize together, were the trimers are then ligated together.

As shown in FIG. 2A, the method of making a “tetramer” includes the following steps: combining a first, second, third and fourth polynucleotide in solution, where at least a portion of first polynucleotide is complementary to at least a portion of the second and fourth polynucleotide, at least a portion of the second polynucleotide is complementary to at least a portion of the first and third polynucleotide and at least a portion of the third polynucleotide is complementary to at least a portion of the second and fourth polynucleotide, and at least a portion of the fourth polynucleotide is complementary to at least a portion of the first and third polynucleotide. The solution is maintained at conditions effective for the first, second, third and fourth polynucleotides associate together to form a tetramer. One or more label molecules are coupled to each tetramer.

A “DNA assembly” is a structure including at least two trimers or two tetramers are associated together, as shown in FIGS. 1B, 5B or FIG. 13B. The DNA assembly can be formed for example, by hybridization of the sticky ends of at least 2 trimers, and subsequently the trimers ligated together. In another aspect, the DNA assembly can be formed, for example, by hybridization of the sticky ends of at least 2 tetramers, as shown in FIG. 12A. In another aspect, the tetramers may be ligated together as shown in FIG. 12B. In some embodiments the assembly is isotropic, and in some embodiments, the assembly is anisotropic thereby providing the ability to link other chemical entities. See FIGS. 1C, 1D and 16-19.

The terms “anneal” and “hybridize” are used interchangeably here and refer to the non-covalent association of complementary strands of polynucleotides, for example the specific association of complementary strands of DNA.

An indication that two nucleic acid sequences are substantially complementary is that the two molecules hybridize to each other under stringent conditions. The phrase “hybridizing specifically to” includes the binding, duplexing, or hybridizing of a molecule to a nucleotide sequence under stringent conditions when that sequence is present in a complex mixture (e.g., total cellular) DNA or RNA.

“Stringent hybridization conditions” and “stringent hybridization wash conditions” in the context of nucleic acid hybridization experiments, such as Southern and Northern hybridizations, are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth et al., Anal. Biochem., 138:267 (1984), which is hereby incorporated by reference in its entirety; Tₘ 81.5° C.+16.6 (log M) +0.41 (% GC) −0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity.

For example, if sequences with >90% identity are sought, the Tₘ, can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ) moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry, and Molecular Biology Hybridization with Nucleic Acid Probes, Part I Chapter 2 “Overview of Principles of Hybridization and the Strategy of Nucleic Acid Probe Assays,” Elsevier, N.Y. (1993), which is hereby incorporated by reference in its entirety. Generally, highly stringent hybridization and wash conditions are selected to be about 500 lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g. more than 100 nucleotides, is 4 6× SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g. >50 nucleotides) Stringent conditions may also be achieved with the addition of destabilizing agents, such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that the encode are substantially identical. This occurs, e.g., when a copy of a nucleic acid is created using the maximum codon degeneracy permitted by the genetic code.

The term “ligation” refers to the process of joining DNA molecules together with covalent bonds. For examples DNA ligation involves creating a phosphodiester bond between the 3′ hydroxyl of one nucleotide and the 5′ phosphate of another. Ligation is preferably carried out at 4-37° C. An presence of a ligase enzyme. Suitable ligases include Thermus thermophilus ligase, Thermus acquaticus ligase, E. coli ligase, T4 ligase, and Pyrococcus ligase.

A nucleic acid assembly may be synthesized following a divergent strategy (growing outwardly from an inner core trimer). A nucleic acid assembly may be synthesized following a convergent strategy (growing inwardly from the outside). See FIG. 2.

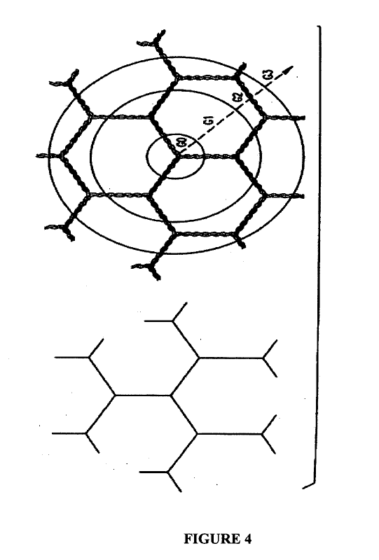

The trimers or tetramers, may be associated together to form nucleic acid assemblies of different shapes. For example, to trimers may be associated together to form a nucleic acid assembly with a “dumbbell” shape. In one embodiment the nucleic acid molecules are DNA. See, FIGS. 2, 3, 12B, 13B, 18 and 19. “Dendrimer-like nucleic acid molecule” (DL-NAM) is a DNA assembly or in other words, DL-DNA. A “honeycomb” structure is a repeating pattern of generally hexagonal structures formed by the association of trimers. See FIG. 1D and 4 (right hand portion). The DNA assembly may also be in the form of a generally linear assembly of trimers, tetramers, or combinations thereof. It will be recognized that the multimers (eg, Y-, X-; T-, dumbbell) are in and of themselves also dendrimer-like nucleic acids.

In the detection molecule, the trimer or tetramers of the invention comprise a specific probe to a target molecule, and also comprise a label or detectable molecule, or detectable reagent, which label, or detectable molecule,, or detectable reagent, include without limitation, chromophores, electrochemical moieties, enzymes, radioactive moieties, phosphorescent groups, fluorescent moieties, chemiluminescent moieties, or quantum dots, or more particularly, radiolabels, fluorophore-labels, quantum dot-labels, chromophore-labels, enzyme-labels, affinity ligand-labels, electromagnetic spin labels, heavy atom labels, probes labeled with nanoparticle light scattering labels or other nanoparticles, fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), epitope tags such as the FLAG or HA epitope enzyme tags such as alkaline phosphatase, horseradish peroxidase, I²-galactosidase, alkaline phosphatase, β-galactosidase, or acetylcholinesterase and hapten conjugates such as digoxigenin or dinitrophenyl, or members of a binding pair that are capable of forming complexes such as streptavidin/biotin, avidin/biotin or an antigen/antibody complex including for example, rabbit IgG and anti-rabbit IgG fluorophores such as umbelliferone, fluorescein, fluorescein isothiocyanate, rhodamine tetramethyl rhodamine, eosin, green fluorescent protein, erythrosin, coumarin, methyl coumarin, pyrene, malachite green, stilbene, lucifer yellow, Cascade Blue, dichlorotriazinylamine fluorescein, dansyl chloride, phycoerythrin, fluorescent lanthanide complexes such as those including Europium and Terbium, Cy3, Cy-5, molecular beacons and fluorescent derivatives thereof, a luminescent material such as luminol; light scattering or plasmon resonant materials such as gold or silver particles or quantum dots; or radioactive material include ¹⁴C, ¹²³I, ¹²⁴I, ¹²⁵I, ¹³¹I, Tc99m, ³⁵S or ³H; or spherical shells and probes labeled with any other signal generating label known to those of skill in the art. For example, detectable molecules include but are not limited to fluorophores as well as others known in the art as described for example, in Principles of Fluorescence Spectroscopy Joseph R. Lakowicz (Editor), Plenum Pub Corp, 2nd edition (July 1999) and the 6^{th} Edition of the Molecular Probes Handbook by Richard P. Hoagland.

Therefore, the detection molecules essentially provide a signal producing system for detection of one or more analytes. The signal producing system may have one or more components, at least one component being a label. A number of signal producing systems may be employed to achieve the objects of the invention. The signal producing system generates a signal that relates to the presence of an analyte (i.e., target molecule) in a sample. The signal producing system may also include all of the reagents required to produce a measurable signal. Other components of the signal producing system may be included in a developer solution and can include substrates, enhancers, activators chemiluminescent compounds, cofactors, inhibitors, scavengers, metal ions, specific binding substances required for binding of signal generating substances, and the like. Other components of the signal producing system may be coenzymes, substances that react with enzyme products, other enzymes and catalysts, and the like. In some embodiments, the signal producing system provides a signal detectable by external means, by use of electromagnetic radiation, desirably by visual examination. exemplary signal-producing systems are described in U.S. Pat. No. 5,508,178.

In addition, non-limiting examples of labels include backbone labels which are nucleic acid stains that bind nucleic acid molecules in a sequence independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); some minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc. Still other examples of nucleic acid stains include the following dyes from Molecular Probes cyanine dyes such as SYTOX Blue, SYTOX Green, SYTOX Orange POPO-1, POPO-3, YOYO-1, YOYO-3, TOTO-1, TOTO-3, JOJO-1, LOLO-1, BOBO-1, BOBO-3 PO-PRO-1, PO-PRO-3, BO-PRO-1, BO-PRO-3, TO-PRO-1, TO-PRO-3, TO-PRO-5, JOPO-PR1, LO-PRO-1, YO-PRO-1, YO-PRO-3. PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61 , -62, -60, -63 (red). Other detectable markers include chemiluminescent and chromogenic molecules, optical or electron density markers, etc.

As noted above in certain embodiments, labels comprise semiconductor nanocrystals such as quantum dots (i.e., Qdots), described in U.S. Pat. No. 6,207,392. Qdots are commercially available from Quantum Dot Corporation. The semiconductor nanocrystals useful in the practice of the, invention include nanocrystals of Group II-VI semiconductors such as MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, and HgTe as well as mixed compositions thereof; as well as nanocrystals of Group III-V semiconductors such as GaAs, InGaAs, InP, and InAs and mixed compositions thereof. The use of Group IV semiconductors such as sermanium or silicon, or the use of organic semiconductors, may also be feasible under certain conditions. The semiconductor nanocrystals may also include alloys comprising two or more semiconductors selected from the group consisting of the above Group III-V compounds, Group II-VI compounds, Group IV elements, and combinations of same.

Formation of nanometer crystals of Group III-V semiconductors is described in U.S. Pat. No. 5,571,018; U.S. Pat. No. 5,505,928; and U.S. Pat. No. 5,262,357, which also describes the formation of Group II-VI semiconductor nanocrystals, and which is also assigned to tie assignee of this invention. Also described therein is the control of the size of the semiconductor nanocrystals during formation using crystal growth terminators. A variety of references summarize the standard classes of chemistry which may be used to link labels to nucleic acid or peptide molecules, such as the “Handbook of Fluorescent Probes and Research Chemicals”, (6th edition) by R. P. Haugland, available from Molecular Probes, Inc., and the book “Bioconjugate Techniques” by Greg Hermanson, available from Academic Press, New York, as well as U.S. Pat. No. 6,207,392.

The semiconductor nanocrystals used in the invention will have a capability of absorbing radiation over a broad wavelength band. This wavelength includes the range from gamma radiation to microwave radiation. In addition, these semiconductor nanocrystals will have a capability of emitting radiation within a narrow wavelength band of about 40 nm or less, preferably about 20 nm or less thus permitting the simultaneous use of a plurality of differently colored semiconductor nanocrystal probes with different semiconductor nanocrystals without overlap i(or with a small amount of overlap) in wavelengths of emitted light when exposed to the same energy source. Both the absorption and emission properties of semiconductor nanocrystals may serve as advantages over dye molecules which have narrow wavelength bands of absorption (e.g. about 30-50 nm) and broad wavelength bands of emission, (e.g. about 100 nm) and broad tails of emission (e.g. another 100 nm) on the red side of the spectrum.

Furthermore, the frequency or wavelength of the narrow wavelength band of light emitted front the semiconductor nanocrystal may be further selected according to the physical properties, such as size, of the semiconductor nanocrystal. The wavelength band of light emitted by the semiconductor nanocrystal, formed using the above embodiment, may be determined by either (1) the size of the core, or (2) the size of the core and the size of the shell depending on the composition of the core and shell of the semiconductor nanocrystal. For example, a nanocrystal composed of a 3 nm core of CdSe and a 2 nm thick shell of CdS will en emit a narrow wavelength band of light with a peak intensity wavelength of 600 nm. In contrast, a nanocrystal composed of a 3 nm core of CdSe and a 2 nm thick shell of ZnS will emit a narrow wavelength band of light with a peak intensity wavelength of 560 nm.

A plurality of alternatives exist for changing the size of the semiconductor nanocrystals in order to selectably manipulate the emission wavelength of semiconductor nanocrystals. These alternatives include: (1) varying the composition of the nanocrystal, and (2) adding plurality of shells around the core of the nanocrystal in the form of concentric shells. It should be noted that different wavelengths can also be obtained in multiple shell type semiconductor nanocrystals by respectively using different semiconductor nanocrystals in different shells, i.e., by not using the same semiconductor nanocrystal in each of the plurality of concentric shells.

Selection of the emission wavelength by varying the composition, or alloy, of the semiconductor nanocrystal is old in the art. As an illustration, when a CdS semiconductor nanocrystal, having an emission wavelength of 400 nm, may be alloyed with a CdSe semiconductor nanocrystal, having an emission wavelength of 530 nm. When a nanocrystal is prepared using an alloy of CdS and CdSe, the wavelength of the emission from a plurality of identically sized nanocrystals may be tuned continuously from400 nm to 530 nm depending on the ratio of S to Se present in the nanocrystal. The ability to select from different emission wavelengths while maintaining the same size of the semiconductor nanocrystal may be important in applications which require the semiconductor nanocrystals to be uniform in size, or for example, an application which requires all semiconductor nanocrystals to have very small dimensions when used in application with steric restrictions.

In some embodiment, the detection molecule comprises a trimer or tetramer nucleic acid molecule(s) linked to semiconductor nanocrystal label, where for example said detection molecule is selected based on its probe component which is specific for the particular detectable substance whose presence or absence, for example, a biological material such as virus or bacterium, is to be ascertained. Thus, the trimer or tetramer is capable of being linked to one or more semiconductor nanocrystal compounds and also capable of specific recognitions of a particular detectable substance in a sample, based on a particular probe component that is also linked to the trimer or tetramer. Of course, in some embodiments, the detection molecule comprises a two or more trimers, tretramers or combinations thereof.

In yet another aspect of the invention, the detection molecules are linked to particular molecule of interest. This feature of the invention is attributed to the structural characteristic of the multimers of the invention, which afford multiple linking points to different molecules or compounds (i.e., multivalency). It follows that depending on the combination of different compounds linked to one or more multimers of the invention, a multimer will exhibit properties with different values when measured in different directions (i.e., anisotropic properties). Put another way, the detection molecules of the invention provide a multiplexing or multiplexed composition for measuring and/or detection of different values.

For example, the various linking sites on a multimer nucleic acid of the invention can be linked to a probes a label, additional nucleic acid molecules (including, siRNA, RNA, antisense DNA, ribozymes, endonucleases, polymerases, peptides, proteins, antibodies, small organic or inorganic molecules, enzyme substrates, ligand/receptors, members of specific binding pairs (e.g., biotin/avidin/streptavidin), or any other molecule that can be linked to the multimers disclosed herein. In other words, the detection molecule provides a multiple platform, onto which, a combination of different molecules are linked (e.g., receptive material). The term “receptive material” includes molecules or compounds that function as a probe, as comprised in a detection molecule, or molecules or compounds positioned on a detection molecule in addition to a probe.

Members of specific binding pairs can be linked to the multimers. Examples of specific binding pairs are replete in the art and include binding partners for an analyte which partners are generally components capable of specific binding to the particular analytes of interest. The binding partner may be a protein which may be an antibody or an antigen. The binding partner may be a member of a specific binding pair (“sbp member”), which is one of two different molecules, having an area on the surface or in a cavity which specific ally binds to and is thereby defined as complementary with a particular spatial and polar organization of the other molecule. The members of the specific binding pair can be members of an immunological pair such as antigen-antibody, although other specific binding pairs such as biotin-avidin, hormones-hormone receptors, enzyme-substrate, nucleic acid duplexes, IgG-protein A. enzyme/substrate, DNA/DNA, oligonucleotide/DNA, chelator/metal, enzyme/inhibitor, bacteria/receptor, virus/receptor, hormone/receptor, DNA/RNA, or RNA/RNA, oligonucleotide/RNA, and binding of these species to any other species, as well as the interaction of these species with inorganic species.

The receptive material that is bound to the multimers is characterized by an ability to specifically bind the analyte or analytes of interest. The variety of materials that can be used as receptive material is limited only by the types of material which will combine selectively (with respect to any chosen sample) with a secondary partner. Subclasses of materials which fall in the overall class of receptive materials include toxins, antibodies, antibody fragments, antigens, hormone receptors, parasites, cells, haptens, metabolites, allergens, nucleic acids nuclear materials, autoantibodies, blood proteins, cellular debris, enzymes, tissue proteins, enzyme substrates, coenzymes, neuron transmitters viruses, viral particles, microorganisms, proteins, polysaccharides, chelators, drugs, and any other member of a specific binding pair, This list only incorporates some of the many different materials that can be coupled onto the trimers or tetramers. Whatever the selected analyte of interest is, the receptive material is designed to bind specifically with the analyte of interest. Other examples of binding pairs that can be incorporated into the detection molecules are disclosed in U.S. Pat. Nos. 6,946,546, 6,967,250, 6,984,491, 7,022,492, 7,026,120, 7,022,529, 7,026,135, 7,033,781, 7,052,854,. 7,052,916 and 7,056,679.

The detection probe can be any member of-a binding pair where one member of the binding pair is the target molecule. Suitable binding pairs include a member of ab antibody-antigen binding pair, a member of a receptor and its corresponding ligand, an aptamer molecule or a member of a pair of complementary nucleic acid molecules. Thus, in one embodiment is where the probe is a ligand or receptor, respectively of a ligand receptor binding pair. In another embodiment, the probe is an antigen or antibody respectively of an antibody-antigen binding pair. In yet another embodiment the probe be a nucleic acid molecule complementary to a target nucleic acid molecule

Therefore a “probe” includes a polynucleotide used for detecting or identifying its corresponding target polynucleotide in a hybridization reaction. The terms “polynucleotide”, “nucleotide”, “nucleotide sequence”, “nucleic acid”, “nucleic acid molecule”, “nucleic acid sequence” and “oligonucleotide” are used interchangeably, and can also include plurals of each respectively depending on the context in which the terms are utilized. Furthermore, such nucleic acids refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA recombinant polynucleotides, branched polynucleotides, plasmids, vectors, isolated DNA (A, B and Z structures) of any sequence, PNA, LNA, TNA (treose nucleic acid), isolated RNA of any sequence, nucleic acid probes, and primers. A polynucleotide may comprise modified nucleotides, such as methylated nucleotides and nucleotide analogs. The sequence of nucleotides may be interrupted by non-nucleotide components.

In yet a further embodiment, the probe can be an aptamer molecule, which do not necessarily bind a similar molecule (eg., DNA not necessarily binding DNA, or peptide not necessarily binding another peptide). Aptamers include DNA, RNA or peptides that are selected based on specific binding properties to a particular molecule. For example, an aptamer(s) can be selected for binding a particular target molecule, whether said, target is nucleic acid, peptide, protein or RNA. Some aptamers having affinity to a specific protein, DNA, amino acid and nucleotides have been described (e.g. K. Y. Wang, et al., “A DNA Aptamer Which Binds to and Inhibits Thrombin Exhibits a New Structural Motif for DNA,” Biochemistry 32:1899-1904 (1993); Pitner et al., U.S. Pat. No. 5,691,145; Gold, et al., “Diversity of Oligonucleotide Function,” Ann. Rev. Biochem. 64: 763-97 (1995); Szostak et al., U.S. Pat. No. 5,631,146).

High affinity and high specificity binding aptamers have been derived from combinatorial libraries. For example, Gold, et at. (U.S. Pat No. 5,270,163) describes the “SELEX” (Systematic Evolution of Ligands by Exponential Enrichment method, where a candidate mixture of single stranded nucleic acid having regions of randomized sequence is contacted with a target molecule (e.g., antigen, peptide or protein fragment). Those nucleic acids having an increased affinity to the target are partitioned from the remainder of the candidate mixture. The partitioned nucleic acids are amplified to yield a ligand enriched mixture. Szostak et al. (U.S. Pat. No. 5,631,146) describes a method for producing a single stranded DNA molecule which binds adenosine or an adenosine-5′-phosphate. Therefore, an aptamer can be selected and utilized as a probe in either the trimers or tetramers.

Furthermore, aptamers may be modified to improve binding specificity or stability as long as the aptamer retains a portion of its ability to bind and recognize its: target monomer. For example, methods for modifying the bases and sugars of nucleotides are known in the art. Typically, phosphodiester linkages exist between the nucleotides of an RNA or DNA. An aptamer according to this invention may have phosphodiester, phosphoroamidite, phosphorothioate or other known lines between its nucleotides to increase its stability provided that the linkage does not substantially interfere witty the interaction of the aptamer with its target monomer.

An aptamer suitable for use in the methods of this invention may be synthesized, by a polymerase chain reaction (PCR), a DNA or RNA polymerase, a chemical reaction or a machine according to standard methods known in the art. For example, an aptamer may be synthesized by an automated DNA synthesizer from Applied Biosystems, Inc. (Foster City, Calif.) using standard chemistries.

In addition, aptamer binding to any desired: target molecule can be optimized post-selection. For example, one modification is “stickiness” of thio- and dithio-phosphate ODN agents to enhance the affinity and specificity to a protein target. In a significant improvement over existing technology, the method of selection concurrently controls and optimizes the total number of thiolated phosphates to decrease nonspecific binding to non-target proteins and to enhance only the specific favorable interactions with the target. Therefore aptamers used in methods of the invention can be modified to permit the selective development of aptamers that have the combined attributes of affinity, specificity and nuclease resistance. Such optimization methods are known in the art, such as in the disclosure of U.S. Pat. No. 6,867,289.

Aptamers may have high affinities, with equilibrium dissociation constants ranging from micromolar to sub-nanomolar depending on the selection used. Aptamers may also exhibit high selectivity), for example, showing a thousand fold discrimination between 7-methylG and G (Haller, A. A., and Sarnow, P., “In Vitro Selection of a 7-Methyl-Guanosine Binding RNA That Inhibits Translation of Capped mRNA molecules, PNAS USA 94.-8521-8526 (1997)) or between D and L-tryptophan (supra, Gold et al.).

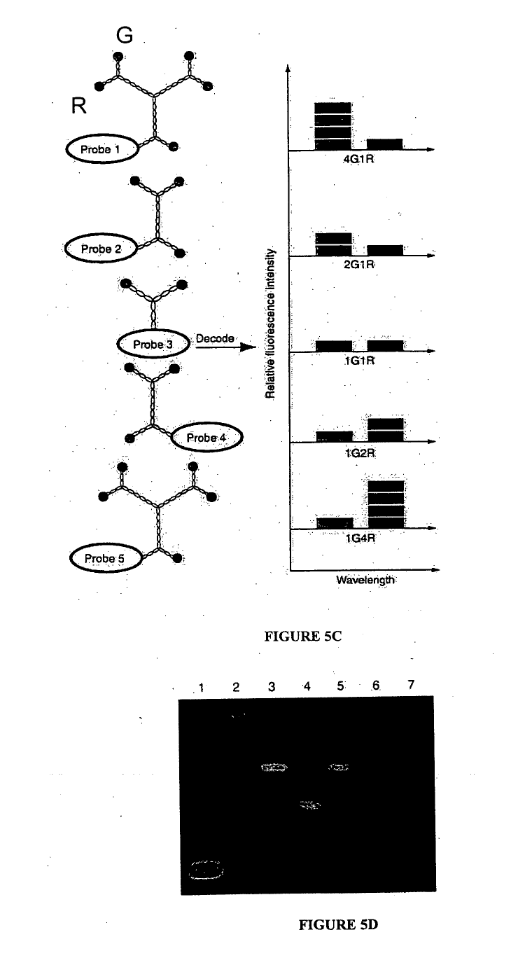

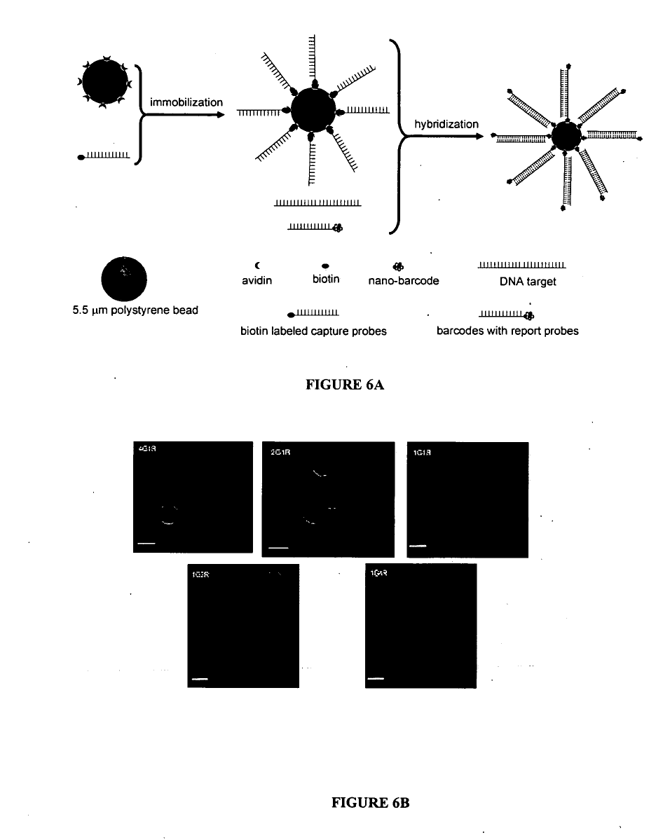

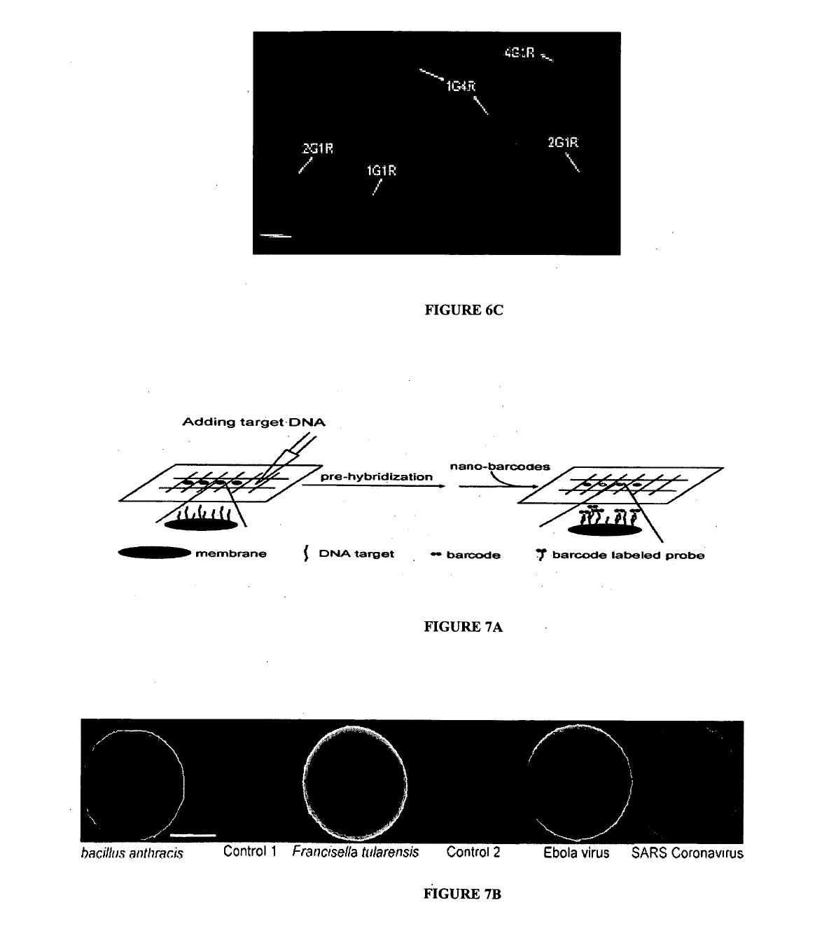

Substrates or solid supports onto which the detection molecules are attacked can be colloidal or planar, including beads, chips films or membranes. e.g., FIGS. 6A, 7A. Examples of beads, chips, membranes, or filters that can be utilized with the detection molecules are replete in the art, such as those disclosed in the relevant parts of U.S. Pat. Nos. 7,045,308, 7,045,283, 7,033,834, 6,949,524 6,982,149, 7,056,704, 7,056,746, 6,917,396, 6,436,561, 6,060,256, 5,988,432, 5,110,216, 7,016,034, 5,750,338 and 5,109,595.

In one embodiment the implementation of assays utilizing the detection molecules are in a planar array format, particularly in the context of biomolecular screening and medical diagnostics, has the advantage of a high degree of parallelity and automation so as to realize high throughput in complex, multi-step analytical protocols. Miniaturization will result in a decrease in pertinent mixing times reflecting the small spatial scale, as well as in a reduction of requisite sample and reagent volumes as well as power requirements. The integration of biochemical analytical techniques into a miniaturized system on the surface of a planar substrate (“chip”), bead substrate, or membrane, will yield substantial improvements in the performance and reduction in cost, of analytical and diagnostic procedures.

Moreover, depending on the desired molecules or combination of molecules linked to the detection molecules of the invention, the substrate effectually provides art array, which can be utilized to measure a variety of different values. The substrate can comprise a plurality detection molecules, which plurality is further comprised of the same or two or more different detection molecules. The term “different” in this context connotes the selection of particular molecules or compounds present on one detection molecule which are different in property, in combination of different types of said particular molecules, or in numbers of said particular molecules. For example, in one embodiment an array comprises a plurality of different detection molecules that are distinguished by the specific target molecule to which they comprise a probe, and distinguished by the number and types of labels present on each or sets of each detection molecule. In other words, it will be appreciated that the array will comprise a subgroup within the plurality that all comprise a probe specific to the same target molecule and thus also comprise the same number/type of a label.

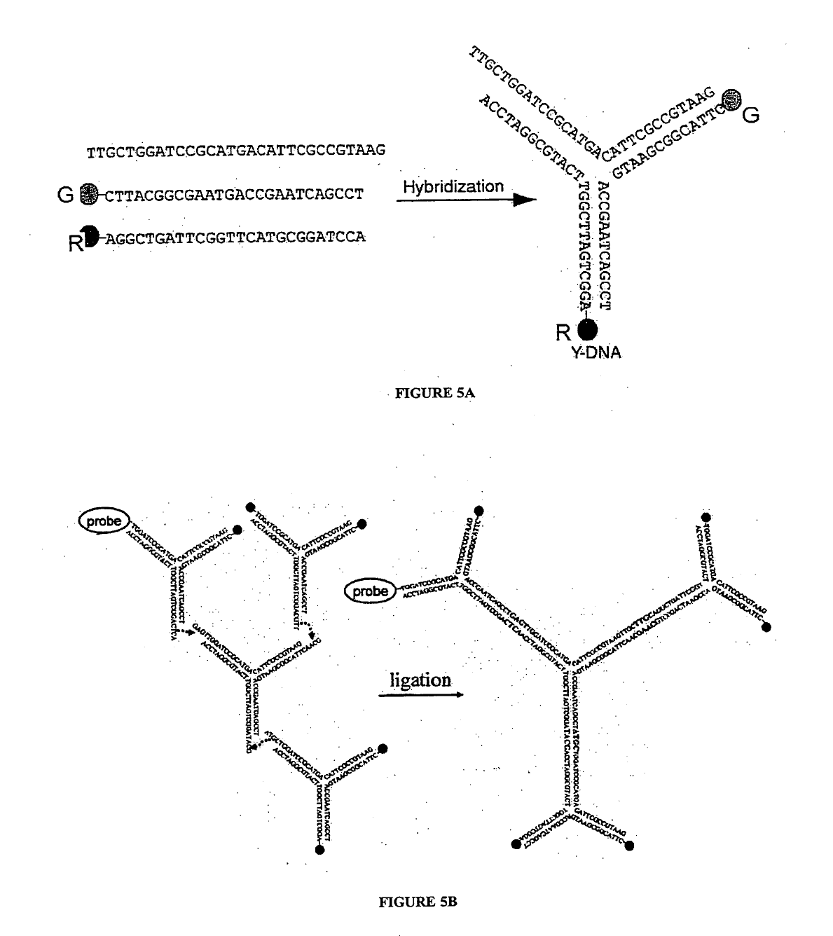

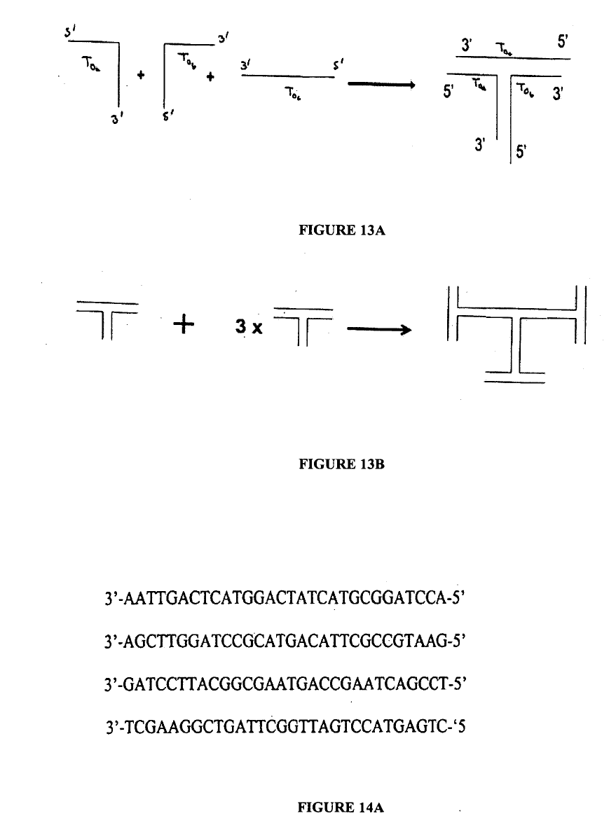

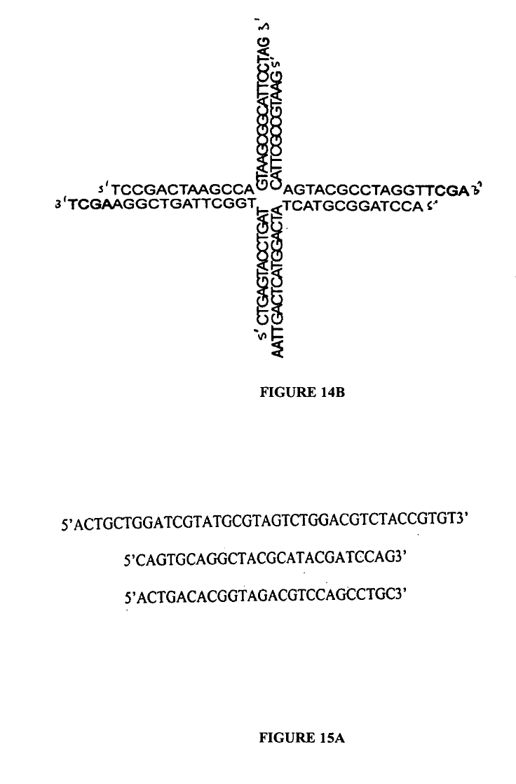

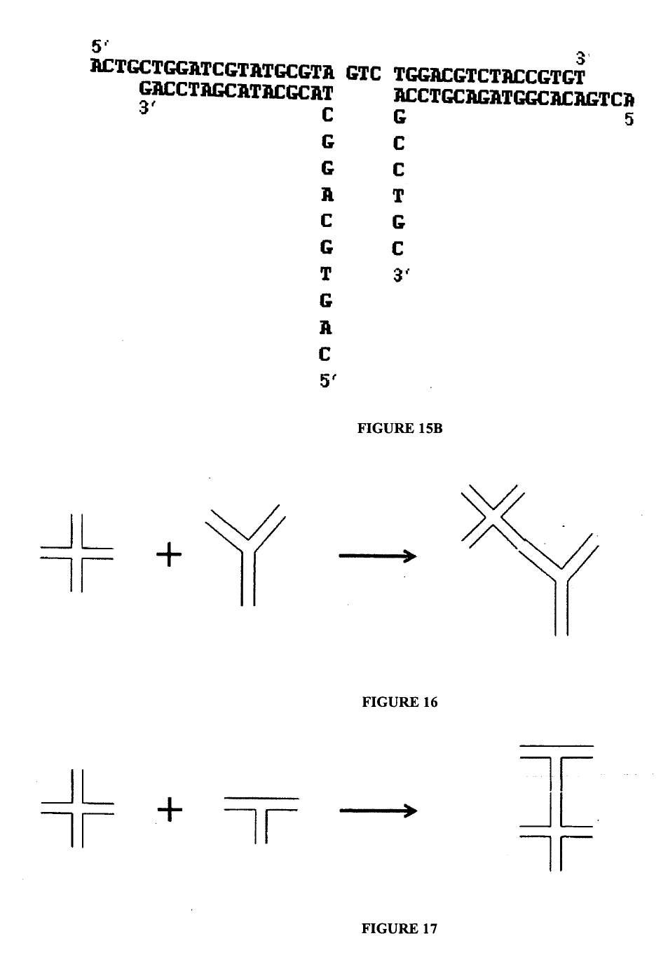

In one embodiment, each trimer or tetramer has a plurality of labels with each label being coupled to a separate polynucleotide, (e.g., FIG. 5A or FIG. 14B). In another embodiments an unlabeled trimer links the trimers having labels. This is shown in FIG. 5B where a first trimer contains the detection probe and a single label, while second and third trimers contain a pair of labels on different polynucleotides. The first, second, and third trimers are coupled together as shown in FIG. 5B with an unlabeled trimer through sticky ends that hybridize to complementary sticky ends.

In, yet another embodiment, an unlabeled tetramer links one or more tetramers having labels. For example, in FIG. 12B, any one of the tetramers being linked to form a multi-unit dendrimer like structure, which can comprise one or more labels. Similarly, in FIG. 13B, any one of the T-shape trimers forming the multi-unit (multi-trimer molecule) can comprise one or more labels. Moreover, after hybridization of any particular pair(s) of polynucleotides, adjoining polynucleotides are ligated together proximate to the sticky ends.

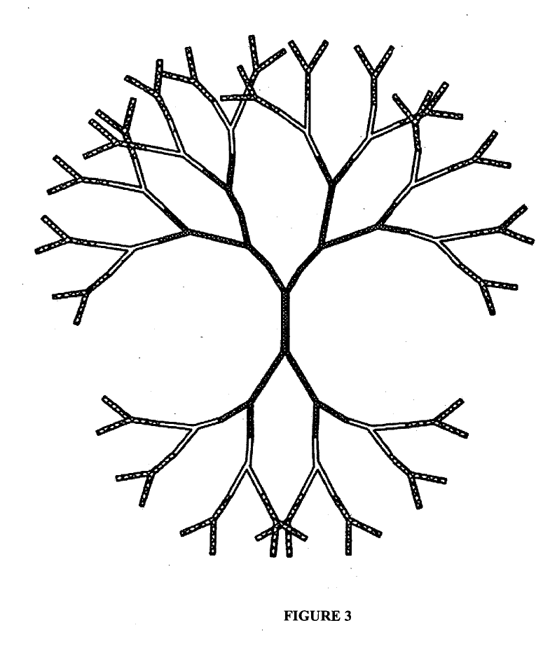

As shown in FIG. 5C, a plurality of detection molecules can be prepared with each having a different probe and a different, label combination. In one embodiment different targets can be detected with only 2 different types of labels. The detection molecules detect and distinguish different target molecules by virtue of different relative fluorescence intensities for the different detection molecules. Other embodiments of the invention are directed to detection molecules comprised of two or more multimers, as depicted in FIGS. 3, 12B, 5B, 13B, and 16-19.

Another aspect of invention relates to a method of detecting a target molecule, if present in a sample. The method includes providing a detection molecule which comprises a probe specific to the target molecule. One or more trimers are connected to the probe, with each trimer comprising a first, a second, and a third polynucleotide. At least a portion of the first polynucleotide is complementary to at least a portion of the second polynucleotide, at least a portion of the first polynucleotide is complementary to at least a portion of the third polynucleotide, and at least a portion of the second polynucleotide is complementary to at least a portion of the third polynucleotide. The first, second, and third polynucleotides are associated together to form a trimer. One or more label molecules are coupled to each trimer. A sample is contacted with the detection molecule under conditions effective to permit target molecules to specifically bind to the probe of the detection molecule. Any specific binding of target molecules to the probe of the detection molecule is detected, thereby detecting the presence of target molecule in the sample.

In one embodiment, in carrying out a method of the invention, a plurality of target molecules, if present in the sample, can be detected with a different detection molecule being used to detect each different target molecule. In this embodiment, each different detection molecule has at least a different probe. In addition, each different detection molecule differs by bow it is labeled. In order to achieve such multiplex detection, where the target molecule is a nucleic acid molecule, the detection probes for each of the detection molecules being utilized must have very similar Tₘ values so that hybridization of each detection molecule to its respective target nucleic acid molecule will occur at substantially the same temperature.

In one embodiment, the contacting step includes contacting the sample with the detection molecule under conditions effective to specifically bind target molecules in the sample to the detection molecule. As a result, a product complex is formed. In this embodiment, the product complex is immobilized on a solid support. In another embodiment the contacting step includes providing a solid support, with one or more capture probes specific to the target molecule. The sample is contacted with the solid support under conditions effective for target molecules in the sample to specifically bind to the capture probes on: the solid support. Next, the detection molecule is contacted with the solid support under conditions effective for the detection molecule to bind to target molecule immobilized on the solid support.

In another embodiment, a capture probe, which separate from a target probe contained on a detection molecule, is linked to a member of a specific binding pair member whereby the cognate member of the specific binding pair is linked to a substrate or solid support. A non-limiting example of such capture probes and specific binding pair members is provided in FIG. 6A, where a capture probe is linked to biotin (and, the solid support comprises avidin) and where hybridization occurs with a target molecule and a reporter molecule comprising a detection molecule.

In another format, the detection molecule is utilized in conjunction with a polymeric microbead. As shown in FIG. 6A, a plurality of capture probes specifically bind to the target molecule. This is achieved by applying avidin to the surface of the polymeric microbead and a capture molecule (in this case, a nucleic acid molecule) which specifically binds to the target molecule is linked to biotin. When the polymeric microbead and biotinylated capture probe are contacted with one another, a plurality of the biotinylated capture molecules are immobilized on the polymeric microbead. The complex of the plurality of biotinylated capture molecules immobilized on the polymeric microbead are contacted with a sample potentially containing the target molecule. The target molecule can then bind to (i.e. hybridize) to the capture molecule. The detection molecule (shown schematically as a reporter probe with nanobarcodes but which collectively take the form of the detection molecule in FIG. 5B) specific for the target molecule will then bind to target molecule immobilized on the microbead.

In an alternative format, the detection molecule is utilized in conjunction with an essentially planar solid support (or substrate). As shown in FIG. 7A, membranes having plurality of immobilized capture probes (in this case, nucleic acid molecules) are fixed on a solid support. This complex of the plurality of capture molecules immobilized on a membrane fixed to a solid support is contacted with a sample potentially containing the target molecule. The target molecule can then bind to (i.e. hybridize) to the capture molecule. The detection molecule (shown schematically as a reporter probe with nanobarcodes but which collectively take the form of the detection molecule in FIG. 5B) specific for the target molecule will then bind to target molecule immobilized on the membrane.

Another aspect of the invention is directed to a library comprising the nanobarcodes (i.e., multimers). The library can comprise any molecular target identifying one or more organisms. The multiplexing feature of the multimers enables detection of numerous different targets simultaneously and from the same sample.

For example, a sample can be screened to determine if multiple different strains of a virus (e.g., influenza virus) is present in the sample. This is achieved by selecting a probe specific for each of multiple strains, such as multiple strains of HIV or influenza virus. Alternatively, the library can target various different pathogens in a sample, such as bacterial pathogens and/or viral pathogens. e.g., Bacillus anthracis (Taton et al., “DNA Array Detection with Nanoparticle Probes,” Science 289: 1757-1760 (2000); Francisella tularensis (Sjostedt et al., a“Detection of Francisella tularensis in Ulcers of Patients with Tularemia by PCR,” J. Clin. Microbiol. 35: 1045-1048 (1997); Ebola virus (Sanchez et al., “Detection and Molecular Characterization of Ebola Viruses Causing Disease in Human and Nonhuman Primates.” J. Infect. Dis. 79:S164-S169 (1999) and SARS Coronavirus (Poon et al, “Detection of SARS Coronavirus in Patients with Severe Acute Respiratory Syndrome by conventional and Real-time Quantitative Reverse Transcription-PCR Assays,” Clin. Chem. 50:67-72 (2004)).

In one embodiment, up to 1000 different codes (i.e., targets) can be identified by utilizing for example, three different colored labels (e.g., Green, Red, Yellow). For example, one probe can contain four molecules of green dye and one of red (e.g., FIG. 5C). Another barcode(i.e., detection molecule) will comprise three molecules of green and two of red label, and so on.

Therefore if a mixture of several probes is added to a solution containing, for example, a bacterial pathogen's DNA, only probes with a particular color code are programmed to bind to that DNA. The results are observed under a fluorescent light microscope using colored filters that pass only one color at a time. A signal in which the ratio of intensity of green light is four times that of red light, for example, identifies a 4G1R” probe. In addition the different ratios of signals produced from a detectable label allow for “decoding” of the nanobarcodes independent of label positioning oil the multimer(s).

In one embodiment, the fluorescence-intensity-encoded nanobarcodes are constructed using fluorescence-labeled trimer or tetramer nucleic acids forming the outer layer of DL-NAM. The detection molecule(s) provides a means of exquisitely controlling both the type and number of labels utilized in the DL-NAMs, thus allowing an extremely effective means to detect and visualize target molecules

Another embodiment is directed to detection of two or more different, target molecules simultaneously with 3 DL-NAM-based nanobarcodes using polystyrene microbeads, where a detection limit is 10⁻¹⁸ mole (attomole) and detection speed is ˜30 seconds. In one embodiment the target molecules are DNA and the DL-NAM comprises DNA.

Samples screened include but are not limited to cells, cell lysate, plasma, buccal or buccal swab, nasal or nasal swab, rectal or rectal swab, throat or throat swab, blood, cell culture medium, culture fluid, cell culture, bodily fluids, amniotic fluid, biopsies, or tissue, fresh, from cells/tissue in culture or from archival cells/tissue, such as frozen samples, Guthrie cards, cord blood, placenta, water soil, air sample gaseous, liquid, food sample, Known methods can be used to obtain a bodily fluid such as blood, sweat, tears, lymph, urine, saliva, semen, cerebrospinal fluid feces or amniotic fluid. Similarly known biopsy methods can be used to obtain cells or tissues such as buccal swab, mouthwash, surgical removal, biopsy aspiration or the like.

The first step in the synthesis of the X-shaped nucleic acid molecule, Y-shaped nucleic acid molecule, T-shaped nucleic acid molecule, and DL-NAM materials is the design and synthesis of specific polynucleotides. The following principles were followed in order to design sequences for DL-NAM (see, eg., N. C., Seeman, “De Novo Design of Sequences for Nucleic Acid Structure Engineering,” Journal of Biomolecular Structure and Dynamics 8:573-581 (1990); N. C. Seeman, “DNA Nanotechnology Novel DNA Constructions,” Annual Review of Biophysics and Biomolecular Structure 27:225-248 (1998), which are hereby incorporated by reference in their entirety).

First, the free energy (deltaG) was calculated far a sequence. In general, a lower free energy is desired. However, intermediate-low deltaG are also considered. Second, the secondary structure of the molecule is considered. In general, the least amount of secondary structure is desired. Third, it needs to be determined if the molecule would form a self-dimer, as it should not form a self-dimer. Fourth, the length is considered, which can vary depending on the design goals. The molecule should be long enough to form stable DNA structure. For X-shaped nucleic acid molecule tetramers, and T-shaped nucleic acid molecule trimers, it should be more than 8 nucleotides (nt) long. Fifth, the helix geometry should be considered. Half-turns should be considered as the quantum of DNA nanostructure. The length between two junctions should be 50% G/C bp, where n is 0, 1, 2, 3 etc. Next, the G/C, content should be considered. In one embodiment, sequences are chosen that constitute about 50% G/C. Last, the symmetry of the molecule should be considered. Sequence symmetry of each aram should be avoided. For nucleic acid molecule sequence design, all oligonucleotides should be checked at the same time. In one embodiment, 4 consecutive nucleotides may be used as a unit in the checking process as follows:

Target sequence: AGCTGAT

Check 1: AGCT. Since no other AGCT sequence appears in that sequence, the first sequence symmetry check passes.

Check 2: GCTG. Since no other GCTG sequence appears in that sequence, the second sequence symmetry check passes.

Check 3: CTGA. Similarly the third sequence symmetry check passes.

Check 4: TGAT. Similarly the fourth sequence symmetry check passes.

In one aspect of the invention, the detection molecule of the invention is a Y-shaped nucleic acid molecule. The specific polynucleotides are combined to form each Y-DNA. Each polynucleotide may include three regions (e.g., see Table 1). A first region (region 1) of each polynucleotide nay include nucleotides that will form a 5′ sticky end when a Y-DNA is formed. A “sticky end” is a single-stranded overhang portion of one of the polynucleotides. In some embodiments, the sticky ends can be 1, 2, 3, 4, 5, 6, 7, 8, 9, or 10 nucleotides. In some embodiments, a polynucleotide may not have this sticky end. In general, a shorter sticky end will allow for less selectivity in binding. For example, a polynucleotide lacking a sticky end would have little to no selectivity. The sticky end in some embodiments is a four nucleotide sticky end. In some embodiments the sticky end includes, or is, TGAC (SEQ ID NO:16), GTCA (SEQ ID NO:17), CGAT (SEQ ID NO:18) ATCG (SEQ ID NO:19), GCAT (SEQ ID NO:20), ATGC (SEQ ID NO:21). TTGC (SEQ ID NO:22), GCAA (SEQ ID NO:23), or GGAT (SEQ ID NO:24) (e.g., Tables 1-12).

The second region (region 2) of each polynucleotide is complementary to the third region (region 3) of one of the other two polynucleotides that form the Y-DNA. The third region of each polynucleotide is complementary to the second region of the other of the other two polynucleotides of Y-DNA. For example, with reference to the sequences in Tables 1 and 2: region 2 of SEQ ID NOs 1-5, represented by SEQ ID NO:25, is complementary to region 3 of SEQ ID) NOs 11-15, represented by SEQ ID NO:30 region 3 of SEQ ID NOs l-5. represented by SEQ ID NO:26 is complementary to region 2 of SEQ ID NOs 6-10, represented by SEQ ID NO:27; and region 2 of SEQ ID NOs 11-15, represented by SEQ ED NO:29, is complementary to region 3 of SEQ ID NOs 6-10 represented by SEQ ID NO:28.

TABLE 1

Sequences of Oligonucleotides

SEQ
ID

| | | | | |
|---|---|---|---|---|
| Strand | NO: | Region 1 | Region 2 | Region 3 |

| | | | | |
|---|---|---|---|---|
| Y₀ₐ | 1 | 5′-TGAC | TGGATCCGCATGA | CATTCGCCGTAAG-3′ |
| Y₁ₐ | 2 | 5′-GTCA | TGGATCCGCATGA | CATTCGCCGTAAG-3′ |
| Y₂ₐ | 3 | 5′-ATCG | TGGATCCGCATGA | CATTCGCCGTAAG-3′ |
| Y₃ₐ | 4 | 5′-ATGC | TGGATCCGCATGA | CATTCGCCGTAAG-3′ |
| Y₄ₐ | 5 | 5′-GCAA | TGGATCCGCATGA | CATTCGCCGTAAG-3′ |
| Y_{0b} | 6 | 5′-TGAC | CTTACGGCGAATG | ACCGAATCAGCCT-3′ |
| Y_{1b} | 7 | 5′-CGAT | CTTACGGCGAATG | ACCGAATCAGCCT-3′ |
| Y_{2b} | 8 | 5′-GCAT | CTTACGGCGAATG | ACCGAATCAGCCT-3′ |
| Y_{3b} | 9 | 5′-TTGC | CTTACGGCGAATG | ACCGAATCAGCCT-3′ |
| Y_{4b} | 10 | 5′-GGAT | CTTACGGCGAATG | ACCGAATCAGCCT-3′ |
| Y_{0c} | 11 | 5′-TGAC | AGGCTGATTCGGT | TCATGCGGATCCA-3′ |
| Y_{1c} | 12 | 5′-CGAT | AGGCTGATTCGGT | TCATGCGGATCCA-3′ |
| Y_{2c} | 13 | 5′-GCAT | AGGCTGATTCGGT | TCATGCGGATCCA-3′ |
| Y_{3c} | 14 | 5′-TTGC | AGGCTGATTCGGT | TCATGCGGATCCA-3′ |
| Y_{4c} | 15 | 5′-GGAT | AGGCTGATTCGGT | TCATGCGGATCCA-3′ |

TABLE 2

Sequence Table

| | |
|---|---|
| SEQ ID NO | Sequence |

| | |
|---|---|
| 1 | 5′-TGACTGGATCCGCATGACATTCGCCGTAAG-3′ |
| 2 | 5′-GTCATGGATCCGCATGACATTCGCCGTAAG-3′ |
| 3 | 5′-ATCGTGGATCCGCATGACATTCGCCGTAAG-3′ |
| 4 | 5′-ATGCTGGATCCGCATGACATTCGCCGTAAG-3′ |
| 5 | 5′-GCAATGGATCCGCATGACATTCGCCGTAAG-3′ |
| 6 | 5′-TGACCTTACGGCGAATGACCGAATCAGCCT-3′ |
| 7 | 5′-CGATCTTACGGCGAATGACCGAATCAGCCT-3′ |
| 8 | 5′-CCATCTTACGGCGAATGACCGAATCAGCCT-3′ |
| 9 | 5′-TTGCCTTACGGCGAATGACCGAATCAGCCT-3′ |
| 10 | 5′-GGATCTTACGGCGAATGACCGAATCAGCCT-3′ |
| 11 | 5′-TGACAGGCTGATTCGGTTCATGCGGATCCA-3′ |
| 12 | 5′-CGATAGGCTGATTCGGTTCATGCGGATCCA-3′ |
| 13 | 5′-GCATAGGCTGATTCGGTTCATGCGGATCCA-3′ |
| 14 | 5′-TTGCAGGCTGATTCGGTTCATGCCGATCCA-3′ |
| 15 | 5′-GGATAGGCTGATTCGGTTCATGCGGATCCA-3′ |
| 16 | 5′-TGAC-3′ |
| 17 | 5′-GTCA-3′ |
| 18 | 5′-CGAT-3′ |
| 19 | 5′-ATCG-3′ |
| 20 | 5′-GCAT-3′ |
| 21 | 5′-ATGC-3′ |
| 22 | 5′-TTGC-3′ |
| 23 | 5′-CCAA-3′ |
| 24 | 5′-GGAT-3′ |
| 25 | 5′-TGGATCCGCATGA-3′ |
| 26 | 5′-CATTCGCCGTAAG-3′ |
| 27 | 5′-CTTACGGCGAATG-3′ |
| 28 | 5′-ACCGAATCAGCCT-3′ |
| 29 | 5′-AGGCTGATTCGGT-3′ |
| 30 | 5′-TCATGCGGATCCA-3′ |

In some embodiments of the invention, the length of each of the regions can vary. For example, in some embodiments, the second and/or third regions are about 13 nucleotides each in length. In some embodiments, the length is of the second and/or third regions may be 7, 8, 9, 10, 11, 12, 13, 14, 15, 16, 17, 18, 19, or 20 nucleotides in length. In some embodiments of the invention, the second and/or third regions may be larger than 20 nucleotides in length, for example they may be about 5, 30, 35, 40, 45, or 50 nucleotides in length.

In one embodiment of the invention, each polynucleotide is 30 nucleotides in length, with the first region having 4 nucleotides, the second region having 13 nucleotides and the third region also having 13 nucleotides. In some embodiments of the invention, the polynucleotides include, essentially include, or are, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, and/or SEQ ID NO:15.

In another aspect of the invention, the detection molecule is comprised of T-shaped or N-shaped nucleic acids. In one embodiment, three specific polynucleotides can be combined to form a T-shaped nucleic acid molecule and four specific polynucleotides are combined to form each X-shaped nucleic acid molecule. T-shaped nucleic acid molecules include 4 regions as shown in Table 3. For X-shaped nucleic acid molecules, each polynucleotide may include three, regions (e.g., Table 5). A first region (region 1) of each polynucleotide may include nucleotides that will form a 5′ sticky end when an X-shaped nucleic acid molecule is formed. A “sticky end” is a single-stranded overhang portion of one of the polynucleotides. In some embodiments, the sticky ends can be 1, 2, 3, 4, 5, 6, 7, 8, 9, or 10 nucleotides. In some embodiments, a polynucleotide may not have this sticky end. In general, a shorter sticky end will allow for less selectivity in binding. For example, a polynucleotide lacking a sticky end would have little to no selectivity. The sticky end in some embodiments is a four nucleotide sticky end. In some embodiments, the sticky end includes, or is, ACTG, CAGT, TCGA, AATT, AGCT, or GATC (e.g., Tables 3-6).

For the T-shaped nucleic acid molecule the second region (region 2) of each polynucleotide is complementary to the fourth region (region 4) of one of the other two polynucleotides. The fourth region of each polynucleotide is complementary to the second region of the other of the other two polynucleotides of T-shaped nucleic acid molecule. The third region is either absent or is a linker to permit information of the T-shaped configuration. For example, with reference to the sequences in Tables 3 and 4: region 2 of SEQ ID NO:33 is complementary to region 4 of SEQ ID NO:31, region 4 of SEQ ID NO:33 is complementary to region 2 of SEQ ID NO:32, and region 2 of SEQ ID NO:31 is complementary to region 4 of SEQ ID NO:32.

For the X-shaped nucleic acid molecule, the region 2 of each polynucleotide is complementary to region 3 of one of the other three polynucleotides. For example, with reference to the sequences in Tables 4 and 5: region 2 of SEQ ID NO: 43 is complementary to region 3 of SEQ ID NO: 46, region 2 of SEQ ID NO: 44 is complementary to region 3 of SEQ ID NO: 43, region 2 of SEQ ID NO: 45 is complementary to region 3 of SEQ ID NO: 44; and region 2 of SEQ ID NO: 46 is complementary to region 3 of SEQ ID NO: 45.

In one embodiment, the length of each of the regions can vary. For example, in some embodiments, the second and/or third regions for the X-shaped nucleic acid molecule aid the second and/or fourth regions of the X-shaped nucleic acid molecules are about 13 nucleotides each in length. In some embodiments, the lengths of these regions may be 7, 8, 9, 10, 11, 12, 13. 14, 15, 16, 17, 18, 19, or 20 nucleotides in length. In some embodiments, these regions may be larger than 20 nucleotides in length, for example they may be about 25, 30, 35, 40, 45, or 50 nucleotides in length.

TABLE 3

Sequences of Oligonucleotides

SEQ

| | | | | |
|---|---|---|---|---|
| ID | Region | Region | Region | Region |

| | | | | | |
|---|---|---|---|---|---|
| Strand | NO: | 1 | 2 | 3 | 4 |

| | | | | | |
|---|---|---|---|---|---|
| T₀ₐ | 31 | 5′-ACTG | CTGGAT | GTC | TGGACGTCTACCGTGT-3′ |
| | | | CGTATG | | |
| | | | CGTA | | |
| T_{0b} | 32 | 5′-CAGT | GCAGGC | | ACGCATACCATCCAG-3′ |
| | | | T | | |
| T_{0c} | 33 | 5′-ACTG | ACACGG | | GCCTGC-3′ |
| | | | TAGACG | | |
| | | | TCCA | | |

TABLE 4

Sequence Table

SEQ
ID

| | |
|---|---|
| NO | Sequence |

| | |
|---|---|
| 31 | 5′-ACTGCTGGATCGTATGCGTAGTCTGGACGTCTACCGTGT-3′ |
| 32 | 5′-CAGTGCAGGCTACGCATACCATCCAG-3′ |
| 33 | 5′-ACTGACACGGTAGACGTCCAGCCTGC-3′ |
| 34 | 5′-ACTG-3 |
| 35 | 5′-CAGT-3′ |
| 36 | 5′-CTGGATCGTATGCGTA′3′ |
| 37 | 5′-GCAGGCT-3′ |
| 38 | 5′-ACACGGTAGACGTCCA-3′ |
| 39 | 5′-GTC-3′ |
| 40 | 5′-TGGACGTCTACCGTGT-3′ |
| 41 | 5′-ACGCATACCATCCAG-3′ |
| 42 | 5′-GCCTGC-3′ |

TABLE 5

Sequences of Oligonucleotides

| | |
|---|---|
| SEQ | |
| ID | Region |

| | | | | |
|---|---|---|---|---|
| Strand | NO: | 1 | Region 2 | Region 3 |

| | | | | |
|---|---|---|---|---|
| X₀ₐ | 43 | 3′-TCGA | AGGCTGATTCGGT | TAGTCCATGAGTC-5′ |
| X_{0b} | 44 | 3′-AATT | GACTCATGGACTA | TCATGCGGATCCA-5′ |
| X_{0c} | 45 | 3′-AGCT | TGGATCCGCATGA | CATTCGCCGTAAG-5′ |
| X_{0d} | 46 | 3′-GATC | CTTACGGCGAATG | ACCGAATCAGCCT-5′ |

TABLE 6

Sequence Table

| | |
|---|---|
| SEQ ID NO | Sequence |

| | |
|---|---|
| 43 | 3′-TCGAAGGCTGATTCGGTTAGTCCATGAGTC-5′ |
| 44 | 3′-AATTGACTCATGGACTATCATGCGGATCCA-5′ |
| 45 | 3′-AGCTTGGATCCGCATGACATTCGCCGTAAG-5′ |
| 46 | 3′-GATCCTTACGGCGAATGACCGAATCAGCCCT-5′ |
| 47 | 3′-TCGA-5′ |
| 48 | 3′-AATT-5′ |
| 49 | 3′-AGCT-5′ |
| 50 | 3′-GATC-5′ |
| 51 | 3′-AGGCTGATTCGGT-5′ |
| 52 | 3′-GACTCATGGACTA-5′ |
| 53 | 3′-TGGATCCGCATGA-5′ |
| 54 | 3′-CTTACGGCGAATG-5′ |
| 55 | 3′-TAGTCCATGAGTC-5′ |
| 56 | 3′-TCATGCGGATCCA-5′ |
| 57 | 3′-CATTCGCCGTAAG-5′ |
| 58 | 3′-ACCGAATCAGCCT-5 |

Therefore in various objects of the invention, the detection molecules comprise polynucleic acid sequences such as DNA, RNA, PNA, TNA or combinations thereof, for example, nucleic acid sequences including those recited in Tables 1-12. In one embodiment, these sequences can be DNA sequences and can be made by methods well known in the art and are useful to prepare the Y-shaped, T-shaped nucleic acid molecule, the X-shaped nucleic acid molecule, and DL-NAM as described herein.

The sequences of the strands, shown in Tables 1 and 3, were designed according to the standards set by Seeman (Seeman, J Biomol Struct Dyn 8-573-81 (1990), which is hereby incorporated by reference in its entirety) and commercially synthesized (Integrated DNA Technologies, Coralville, Iowa). All oligonucleotides were dissolved in annealing buffer (10 mM Tris, pH 8.0, 50 mM NaCl, 1 mM EDT) with a final concentration of 0.1 mM incubated at 95° C. for 2 min., quickly cooled to 60° C., and slowly cooled to 4° C., as follows:

Annealing Program:

| | Control | block |
|---|---|---|
| | Lid | 105° C. |
| (Denaturation) | 95° C. | 2 min |
| (Cooling) | 65° C. | 2 min |
| (Annealing) | 60° C. | 5 min |
| (Annealing) | 60° C. | 0.5 min |
| | Temperature increment −1° C. | |
| (number of cycle) | go to (5) Rep 40 | |
| (Hold) | 4° C. | enter |

X-shaped nucleic acid molecules can be synthesized by mixing equal amounts of four oligonucleotide strands. The nomenclature is as follows: X₀ₐ, X_{0b}, X₀ₑ, and X_{0d} are the four corresponding single oligonucleotide chains that form a X₀ nucleic acid molecule (X₀). Similarly, X₁ₐ, X_{1b}, X_{1c}, and X_{1d} are the four corresponding single oligonucleotide chains that form an X₁-nucleic acid molecule (X₁); and Xₙₐ, X_{nb}, X_{nc}, and X_{nd} are the four corresponding single oligonucleotide chains that form a Xₙ-shaped nucleic acid molecule (Xₙ). The reactions can be the following: X₀ₐ+X_{0b}+X_{0c}+X_{0d}, X₁ₐ+X_{1b}+X_{1c}+X_{1d}→X₁, and Xₙₐ+X_{nb}+X_{nc}+X_{nd}→Xₙ, etc. (see FIGS. 12A-3B).

T-shaped nucleic acid molecules can be synthesized by mixing equal amounts of three oligonucleotide strands. The nomenclature is as follows: T₀ₐ, T_{0b}, and T_{0c} are three corresponding single oligonucleotide chains that form a T₀-nucleic acid molecule (T₀). Similarly, T₁ₐ, T_{1b}, and T_{1c} are the three corresponding single oligonucleotide chains that form a T₁-nucleic acid molecule (T₁); and Tₙₐ, T_{nb}, and T_{nc} are the three corresponding single oligonucleotide chains that form a Tₐ-shaped nucleic acid molecule (Tₙ). The reactions can be the following: T₀ₐ+T_{0b}+T_{0c}→T₀, T₁ₐ+T_{1b}+T_{1c}→T₁, and T₁, and Tₙₐ+T_{nb}+T_{nc}→Tₙ, etc. (see FIGS. 13A-B).

Y-shaped nucleic acid trimers can be synthesized by mixing equal amounts of three oligonucleotide strands. The nomenclature is as follows: Y₀ₐ, Y_{0b}, and Y_{0c} are the three corresponding single oligonucleotide chains that form a Y₀-nucleic acid (Y₀). Similarly, Y₁ₐ, Y_{1b}, and Y_{1c} are the three corresponding single oligonucleotide chains that form a Y₁-nucleic acid (Y₁); and Yₙₐ, Y_{nb}, and Y_{nc} are the three corresponding single oligonucleotide chains that form a Yₙ-nucleic acid (Yₙ). The reactions can be the following: Y₀ₐ+Y_{0b}+Y_{0c}→Y₀, Y₁ₐ+Y_{1b}+Y_{1c}→Y₁, and Yₙₐ+Y_{nb}+Y_{nc}→Yₙ, etc. (see FIGS. 1A-1B). The production of Y-shaped nucleic acid molecules and their assembly into DL-NAM is described more fully in U.S. patent application Ser. No. 10/877,697, which is hereby incorporated by reference in its entirety. In addition, production of X-shaped or T-shaped nucleic acids and their assembly into DL-NAM is described more fully in U.S. Provisional Application Ser. No. 60/756,453, which is hereby incorporated by reference in its entirety.

X-shaped nucleic acids, T-shaped nucleic acids, and Y-shaped nucleic acids are the basic building blocks for the detection molecules. Two strategies can be adopted to synthesize the X-shaped nucleic acids, T-shaped nucleic acids, and Y-shaped nucleic acids: stepwise and all-in-one. In the stepwise approach, two oligonucleotides with complementary regions formed one arm of a X-shaped nucleic, acids, T-shaped nucleic acids, and Y-shaped nucleic acids are placed in contact with each other; then a third oligonucleotide, that is complementary to the first two un-matched regions of oligonucleotides of the T-shaped or Y-shaped nucleic acids (or, in the case of N-shaped nucleic acids, complementary to one of the un-matched regions of oligonucleotides) is added to form other two arms of the T-shaped nucleic acids and Y-shaped nucleic acids (and one other arm of the X-shaped nucleic acids) and, only in the case of X-shaped nucleic acid, a fourth oligonucleotide that is complementary to the two remaining un-matched regions of oligonucleotides is added to form an X-shaped tetramer. In the all-in-one approach, all three or four oligonucleotides are mixed together in equal amounts to form, respectively, T-shaped nucleic acids, Y-shaped nucleic acids, or the X-shaped nucleic acids.

In some embodiments, the detection molecules (e.g., barcodes or nanobarcodes) are VDL-NAM, whereby individual X-shaped nucleic acid molecules are ligated specifically to other X-shaped nucleic acid molecules without self-ligation. The ligations can be performed with Fast-Link DNA Ligase (Epicentre Technologies, Madison, Mich.). T4 DNA ligase may also be used (Promega Corporation, Madison, Wis.). FIG. 1B. The nomenclature of DL-NAM is as follows: the core of the dendrimer, X₀, is designated as G₀, the 0 generation of DL-NAM. After X₀ is ligated with X₁. the dendrimer is termed the 1^{st} generation of DL-NAM (G₁), and so on. The n^{th} generation of DL-NAM is noted as Gₙ.

X-shaped nucleic acid molecules are composed of four single DNA strands. These strands are designed so that ligations between Xᵢ and Xⱼ can only occur when i≠j (no self-ligation). In addition, the ligation can only occur in one direction, that is, X₀→X₁→X₂→X₃→X₄. In other words, X₀ is ligated to X₁ with 1-4 stoichiometry four X₁ units are linked with one X₀ forming 1^{st} generation of DL-NAM (G1). G1 can then be ligated to eight X₂ units due to the fact that there are eight arms of X₁ now (each X₁ posses two arms), and the resulting product is a second-generation DL-NAM (G₂). A third (G₃), fourth (G₄), and even higher generation DL-NAM could be synthesized in a similar way. The resulting dendrimers (Gₙ) have only one possible conformation due to the designed unidirectional ligations. The general format on the n^{th} generation DL-NAM is Gₙ=(X₀)(3X₁)(6X₂) . . . (4×2ⁿ⁻¹Xₙ), where n is the generation number and Xₙ is the n^{th} X-shaped nucleic acid molecule. The total number of X-DNA in an nth generation DL-NAM are 4×2ⁿ⁻¹−2. Therefore, the growth of DL-NAM from n^{th} generation to (n+1)^{th} generation requires a total of 3×2ⁿ new Xₙ₊₁-nucleic acid molecule.

The first generation DL-NAM can be built by ligating X₀ and X₁ with 1:3 stoichiometry. The ligation product migrates as a single band, and its mobility is slower than that of its building block, X₀. The presence of a single band indicates that a new molecular species with a well-defined stoichiometry is formed. The estimated yield is more than 95%.

To further evaluate the structure of the ligation product, it can be denatured and examined by gel electrophoresis. There are two major band, for the denatured sample—one with the same mobility as the single strand X_{0b} (30-mer) and one with slower mobility (see arrow, a single stranded 90-mer strand), which is exactly what one would expect according to the assembly scheme. Denaturing G₁ DL-NAM results in two sizes of single strands left: one 30-mer (X_{1b}) and the other 90-mer ((X₁ₐ)(X₀ₐ)(X_{1c}), (X₁ₐ)(X_{0b})(X_{1c}), and (X₁ₐ)(X_{0c})(X₁c)). Taken together, these results indicate that the formation of the 1^{st} generation of DL-NAM would be expected with a high yield.

The second, third, and fourth generation DL-NAM can be synthesized with the stepwise approach and evaluated by get electrophoresis. With each increased generation, the mobility of the ligated product will decrease. The yield and the purity of higher generations (G₃ and G₄) DL-NAM will not decrease, even without purification, because the stepwise synthesis approach is very robust.

Similar procedures can be used to form dendrimeric structures from T-shaped trimers, as shown in FIGS. 17-19.

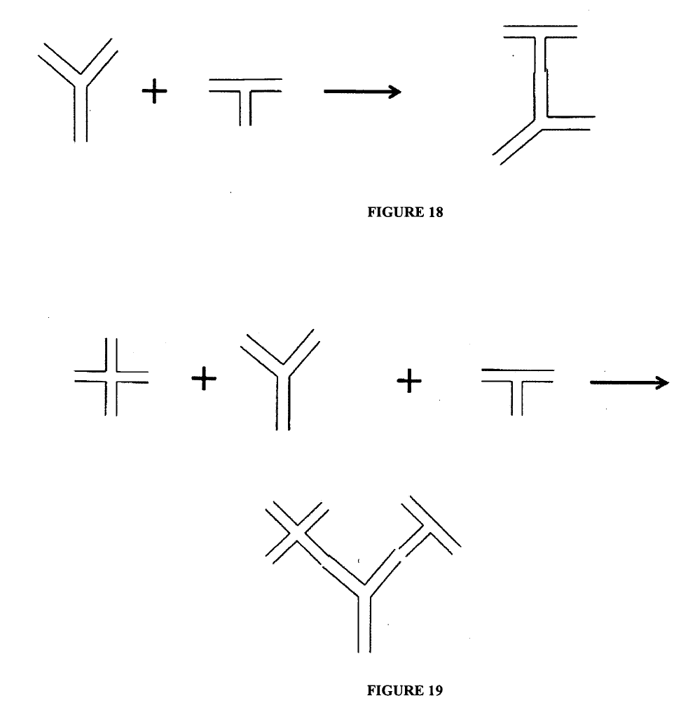

In addition, combinations of X-shaped nucleic acid molecules, T-shaped nucleic acid molecules, and Y-shaped nucleic acid molecules can be used to make dendrimeric structures in substantially the same manner as described above. For example, the combination of X-shaped nucleic acid molecules and Y-shaped nucleic acid molecules is shown in FIG. 16. Such combinations of different multimers or even the same multimers result in dendrimer or dendrimer like structures. FIGS. 17-19. T-shaped nucleic acid molecules and Y-shaped nucleic acid molecules can be combined in accordance with FIG. 18. Finally, X-shaped nucleic acid molecules, T-shaped nucleic acid molecules, and Y-shaped nucleic acid molecules are combinable, as shown in FIG. 19, to produce dendrimeric structures.

### EXAMPLES

### Example 1

### Synthesis of DNA Nanobarcodes

Dendrimer-like nucleic acid molecule (DL-NAM) nanostructures were constructed as described herein (Supra, Li et al. 2004).

The multivalent and anisotropic properties of DL-NAM were utilized here as fluorescent dye carriers (i.e. scaffoldings) to construct fluorescence-intensity-encoded nanobarcodes. Fluorescence labeled Y-shaped DNA (Y-DNA molecules were first synthesized where each Y-DNA consisted of three oligonucleotide components that are complementary to each other. One of the oligonucleotides consisted of a sticky end, and the other two were labeled with either fluorophores or a molecular probe. After hybridization these oligonucleotides formed a fluorescence labelled Y-DNA (FIG. 5A) that was used as a peripheral outmost layer of DL-NAM to construct fluorescence labeled DNA nanostructures. Since both dye type and dye number can be precisely controlled, multicolor fluorescence-intensity-encoded nanobarcodes could be fabricated (FIG. 5B). The decoding was determined by the ratio of different fluorescent dyes, independent of the dye positions (FIG. 5C). The coding capacity (C) is calculated by the following formula:

$$C = \frac{( P + L - 1 ) !}{P ! ( L - 1 ) !} ,$$

where L is the color number and P is the labelled position number, which is determined by the generation number of DL-NAM. For example more than one thousand different nanobarcodes can be fabricated with three fluorescent colors and with a third generation (G₃) DL-NAM as fluoro-dye carriers. The actual available codes, of course, can be much fewer depending on the number of targets, equipment sensitivity and signal-to-noise requirement. During the construction of DNA nanobarcodes, the molecular probes were linked to the free reactive ends of DL-NAM. A myriad of DNA manipulation enzyme tools (Luo, D., “The Road From Biology to Materials,” Materials Today Vol. 6, pp. 38-43 (2003), which is hereby incorporated by reference in its entirety) makes it very easy to attach molecular probes (e.g., DNA or PNA probes, or even antibodies) to DNA nanobarcodes. Consequently the resultant nanobarcodes, which were built entirely from DNA molecules, not only had coding capacity, but also had molecular recognition elements, which could be used for molecular detection,

Each barcode building block, the fluorescence labelled Y-DNA, consisted of three oligonucleotides (Table 1 and 2), one of which contained a non-palindromic sticky end white the oiler two were either fluorescence labelled or attached with a DNA probe.

TABLE 7

Building Oligonucelotides

SEQ
ID

| | | | |
|---|---|---|---|
| Strand | NO. | Segment 1 | Segment 2 |

| | | | |
|---|---|---|---|
| y1g | 59 | 5′/Phos/TTGC | TGGATCCGCATGACATTCGCCGTA |
| | | | AG-3′ |
| y1h | 60 | 5′/Phos/CGTT | TGGATCCGCATGACATTCGCCGTA |
| | | | AG-3′ |
| y1d | 61 | 5′/Phos/ATGC | TGGATCCGCATGACATTCGCCGTA |
| | | | AG-3′ |
| y1Alex488 | 62 | 5′/Alex488/ | TGGATCCGCATGACATTCGCCGTA |
| | | | AG-3′ |
| y2D | 63 | 5′/Phos/GCAT | CTTACGGCGAATGACCGAATCAGC |
| | | | CT-3′ |
| y2e | 64 | 5′/Phos/GCAA | CTTACGGCGAATGACCGAATCAGC |
| | | | CT-3′ |
| y2Alex488 | 65 | 5′/Alex488/ | CTTACGGCGAATGACCGAATCAGC |
| | | | CT-3′ |
| y2BO630 | 66 | 5′/BO630/ | CTTACGGCGAATGACCGAATCAGC |
| | | | CT-3′ |
| y3D | 67 | 5′/Phos/GCAT | AGGCTGATTCGGTTCATGCGGATC |
| | | | CA-3′ |
| y3E | 68 | 5′/Phos/TTGC | AGGCTGATTCGGTTCATGCGGATC |
| | | | CA-3′ |
| y3H | 69 | 5′/Phos/AACG | AGGCTGATTCGGTTCATGCGGATC |
| | | | CA-3′ |
| y3BO630 | 70 | 5′/BO630/ | AGGCTGATTCGGTTCATGCGGATC |
| | | | CA-3′ |

Notes:
(1) 5′/Phos/ indicates 5′ Phosphorylation modification
(2) 5′/Alex488/ indicates 5′ Alexa Fluor 488 NHS Ester modification
(3) 5′/BO630/ indicates 5′ Bodipy 630/650-X NHS Ester modification

TABLE 8

Capture Probes, Report Probes, and Target
DNA

SEQ
ID

| | | | |
|---|---|---|---|
| NO. | strand | Segment 1 | Segment 2 |

| | | | | |
|---|---|---|---|---|
| Capture | 71 | Capture-Ban | 5′ biotin | ATC CTT ATC |
| probe | | | | AAT AT |
| | 72 | Capture-Ebo | | TGG TGG GTT |
| | | | | ATA AT |
| | 73 | Capture-Sco | | C CTG TGA ACC |
| | | | | AAG |
| | 74 | Capture-SMPo | | CGT CTC TAC |
| | | | | CTG AT |
| | 75 | Capture- | | AAT TAA CAA |
| | | control | | TAA |
| y1- | 76 | y1-report-Ban | 5′ T TAA | TGGATCCGCATGAC |
| report | | | CAA TAA | ATTCGCCGTAAG- |
| probe | | | | |
| | 77 | y1-report-Ebo | 5′ A ATC | 3′ |
| | | | ACT GAC | |
| | | | ATG | |
| | 78 | y1-report-Sco | 5′ ACG | |
| | | | CAG TAT | |
| | | | TAT | |
| | 79 | y1-report-SMPo | 5′ T ACT | |
| | | | ATT GCA | |
| | | | TCT | |
| | 80 | y1-report- | GGA TTA | |
| | | control | TTG TTA | |
| | | | ATT | |

| | | | |
|---|---|---|---|
| Target | 81 | Target 1 | 5′ GGA TTA TTG TTA AAT |
| DNA | | (B. anthracis) | ATT GAT AAG GAT |
| | 82 | Target 2 | 5′ CAT GTC AGT GAT TAT |
| | | (F. tularensis) | TAT AAC CCA CCA |
| | 83 | Target 3 | 5′ ATA ATA CTG CGT CTT |
| | | (Ebola virus) | GGT TCA CAG C |
| | 84 | Target 4 | 5′ AGA TGC AAT AGT AAT |
| | | (SARS Coronavirus) | CAG GTA GAG ACG |

Two types of fluorescence dyes, Alexa Fluor 488 (Ex=495 nm and Em=519 nm) and BODIPY 630/650 (Ex=625 nm and Em=640 nm), were used to label DNA (Table 1 and 2). The fluorescence-labelled Y-DNA (Table 3) were ligated to other Y-DNA via complementary sticky ends.

TABLE 9
Y-DNA Building Blocks

| Oligonucleotide | Hybridization | Y-DNA |
|---|---|---|
| y1h + y2e + y3D | => | Y-core |
| y1-probe + y1Alex488 + y3BO630 | | YGR-probe1 |
| y1-probe + y2Alex488 + y3D | | YG-probe2 |
| y1-probe + y2Alex488 + y3H | | YG-probe3 |
| y1Alex488 + y2Alex488 + y3E | | YGG |
| y1d + y2Alex488 + y3BO630 | | YGR1 |
| y1Alex488 + y2BO630 + y3E | | YGR2 |
| y1d + y2BO630 + y3BO630 | | YRR |
| y1-probe + y2BO630 + y3H | | YR-probe4 |
| y1-probe + y2D + y3BO630 | | YR-probe5 |

Five nanobarcodes, 4GR1R, 2G1R, 1G2R, 1G2R and 1G4R, where the number refers to the quantity of each dye molecule on one barcode (for example, there were 4 green dyes and 1 red dye on 4G1R), were constructed (Table 4 and FIG. 5C).

TABLE 10
DNA Nanobarcodes

| | | DL-NAM based |
|---|---|---|
| Y-DNA building blocks | ligation | nanobarcodes |
| Y-core + YG-probe3 + YGG + YGR1 | => | 4G1R |
| YG-probe2 + YGR1 | | 2G1R |
| YGR-probe1 | | 1G1R |
| YR-probe5 + YGR1 | | 1G2R |
| Y-core + YR-probe4 + YRR + YGR2 | | 1G4R |

The three DNA components were complementary to one half of each other. After hybridization, they formed Y-DNA which was used as the outermost peripheral layer of the nanobarcodes. Other non-fluorescence labeled Y-DNA was used to link fluorescence-labeled Y-DNA together. All Y-DNA were ligated to each other via their complementary sticky ends to form fluorescence-labeled dendritic nanostructures (nanobarcodes) (FIG. 5B).

### Example 2

### Gel Electrophoresis

The DNA nanobarcodes were run in a 3% agarose ready gel (Bio-Rad. Hercules, Calif.) at 85 volts at room temperature in Tris-acetate-EDTA (TAE) buffer (40 mM Tris, 20 mM Acetic Acid and 1 mM EDTA, pH 8.0, Bio-Rad, Hercules, Calif.). After a true color picture of the gel was taken using a digital camera under strong UV illumination, it was stained with 0.5 μg/ml of ethidium bromide in Tae buffer. Briefly, 10 pmol of DNA sample in a denaturing buffer (10 mM EDTA. 25 mM NaOH) was heated at 95° C. for 2 min and then immediately cooled down in a −20° C. freezer. The denatured DNA sample was run through a 3% agarose gel at 50 v for 10 min and then 100 v for 80 min at 4° C. in TAE buffer containing 0.5 μg/ml of ethidium bromide.

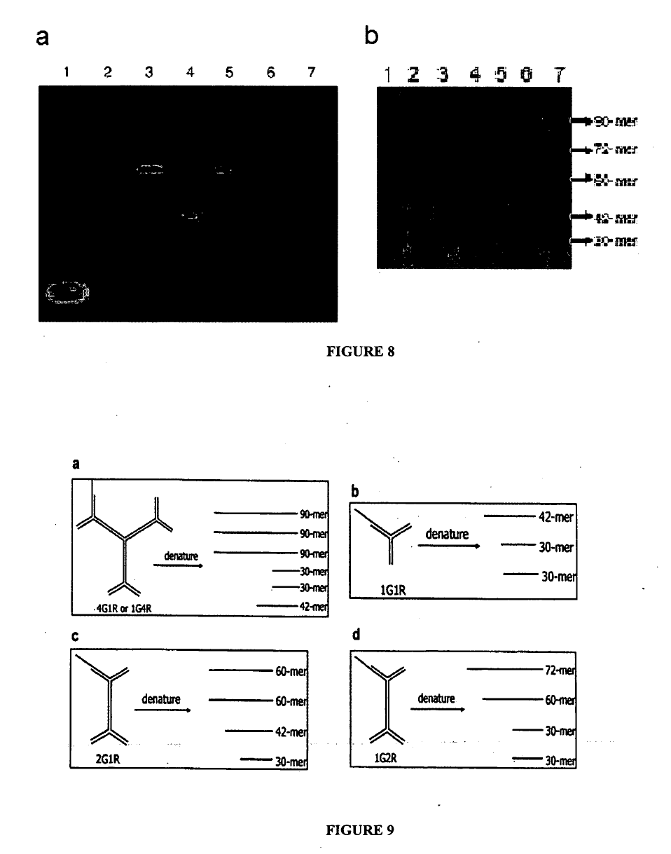

With Alexa Fluor 488 alone and BODIPY 630/650 alone labelled oligonucleotides as controls (FIG. 5D, lanes 1 and 7, respectively), the obvious color changes from green and yellow to red (FIG. 5D, lanes 2 to 6) indicated the formation of the expected different nanobarcodes, which was further confirmed by the electrophoretic mobility shift of DNA nanobarcodes relative to the starting oligonucleotides (FIG. 5D and FIG. 8A). The formation of DNA nanobarcodes with the desired, dendritic architectures was also confirmed by the generation of oligonucleotides with new length, which were revealed by denaturing agarose gel electrophoresis (FIG. 8B and FIG. 9).

### Example 3

### Library

To detect pathogens (here, Bacillus anthracis, Francisella tularensis, Ebola virus, and SARS Coronavirus were targeted), a small fragment of characteristic DNA sequences from each potential species' genome was selected as the target DNA. Two separate sets of DNA probes, which were complementary to the two regions of the same target DNA, were synthesized. One blank control where the two sets of probes were complementary to each other, was also chosen. Thus, a library (Table 5) of two sets of single stranded DNA probe (Table 2) was created.

TABLE 11

Code Library

| | |
|---|---|
| barcode | Coded target |

| | |
|---|---|
| 4G1R | Anthrax lethal factor of bacillus |
| | anthracis |
| | (GGA TTA TTG TTA AAT ATT GAT AAG GAT) |
| | (SEQ ID NO:81) |
| 2G1R | Lipoprotein gene of Francisella tularensis |
| | (435-463) |
| | (GCT GTA TCA TCA TTT AAT AAA CTG CTG) |
| | (SEQ ID NO:82) |
| 1G1R | L gene of Ebola virus (13601-13631) |
| | (CAT GTC AGT GAT TAT TAT AAC CCA CCA) |
| | (SEQ ID NO:83) |
| 1G2R | Control, where the capture probe and the |
| | report were complementary to each other |
| | for hybridization control |
| 1G4R | N gene of SARS Coronavirus (28262-28286) |
| | (ATA ATA CTG CGT CTT GGT TCA CAG C) |
| | (SEQ ID NO:84) |

One set of probes (capture probes) was biotin-labeled and complementary to one part of their own target DNA. The other set of probes (report probes), which was complementary to the other part of the target DNA, was attached to the nanobarcodes.

### Example 4

### Microbead Based Molecular Detection

The diameter of DNA nanobarcodes was less than 30 nm, which is far beyond the detection limit of optical microscopy. Polystyrene microbeads (diameter=5.5 μm) were thus employed to amplify the fluorescence signals for imaging and molecular detection.

The conjugates between microbeads and DNA probes were prepared using a modified protocol suggested by the microbead manufacturer (Bangs Laboratories Inc., Fishers, Ind.). Briefly, 1.0 μg of streptavidin-coated polystyrene microbead suspension was washed with 100 μl of TTL buffer (100 mM Tris-HCl pH 8.0, 0.01% Tween 20) 1M LiCl) and re-suspended in 10 μl of TTL. One picomole of biotin-modified capture probes was then mixed with the microbead suspension and incubated at room temperature with gentle agitation for 30 minutes. After that, the excess and weakly bound probes were removed using sequential washes with 100 μl of TTL buffer. TT buffer (250 mM Tris-HCl, pH 8.0 0.01% Tween 20), TTE buffer (250 mM Tris-HCl, pH 8.0, 0.01% Tween 20, 20 nM Na₂(EDTA)), and TT buffer. The probe-functionalized Microbeads were re-suspended in pre-hybridization buffer (Church buffer:0.5 M sodium phospate pH 8.0 1 mM EDTA, 7% (w/v) SDS and 1% (w/v bovine serum album) and incubated at 68° C. for 30 minutes. After the pre-hybridization buffer was removed, the microbeads were re-suspended in hybridization buffer (1×SSC (150 mM sodium chloride, 15 mM sodium citrate), 1% SDS). Other DNA probes were conjugated to microbeads as described above.

The microbeads used for flow cytometer analysis were purposely prepared non-uniformly in terms of the number of target DNA bound to each microbead (since only a small amount of target DNA wag used, microbeads were far from saturation in terms of barcode binding). Bead-probe conjugates, along with a sample containing “unknown” DNA target(s) and nanobarcodes were added into 400 μl of hybridization buffer individually without mixing in order to achieve a non-uniform, barcode binding. The resultants microbead suspension was incubated at room temperature in the dark for 2 hours.

The sample was then analyzed using a flow cytometer (BD FACSCalibur) with green (FL1H) and far-red channels (FL4H). On the other hand, the sample for fluorescence microscopy analysis was prepared by thoroughly mixing bead/probe conjugates along with a sample containing “unknown” targets) and nanobarcodes in 400 μl of hybridization buffer (so that binding will be uniform). The hybridization was performed with gentle agitation. The sample was then washed with 400 μl of hybridization buffer three times to remove excess and weakly bound nanobarcodes. Subsequently, the sample was imaged with fluorescence microscopy, and the images were analyzed by Metamorph software.

The microbead-based amplification strategy and detection format are shown in FIG. 6A. In particular, two sets of single-stranded DNA (ssDNA) probes were used. One set of probes (capture probes) was biotin-labelled first and then was immobilized onto avidin-functionalized microbeads. Note that each batch of microbeads consisted of only one type of capture probe (complementary to a particular target DNA, here “target DNA” refers to sample DNA to be detected). Multiple types of microbeads were then pooled together after biotin-avidin conjugations to form a library of microbeads. The other set of probes (report probes), on the other hand, was coupled to specific nanobarcodes where each report probe was designed to be complimentary to another part of a particular target DNA and thus was able to be hybridized onto a specific microbead via a sandwiched hybridization in the format of “bead-capture probes-target DNA-report probes-nanbarcodes” (FIG. 6A) As a result of each microbead binding to a large amount of sandwiched complexes, fluorescence signals are amplified. In the assay, the sample containing unknown DNA molecules (i.e., target DNA) was mixed with microbeads in suspension, and target DNA was captured onto a particular type of microbeads through complementary annealing to the corresponding capture probes. The solution containing a barcode library was then added.

Since each barcode was connected with a particular report probe which in turn hybridized to another portion of the target DNA, nano barcodes were specifically bound to corresponding microbeads. The resultant barcode attached microbeads were first evaluated individually by fluorescence microscopy, and the overlay color (pseudocolor) images are shown in FIG. 6B. Such pseudocoloring imaging allows visualization of several different probes based on fluorescence detection of different combinations of fluorescence colors (e.g., Red and Green), FIG. 6B.

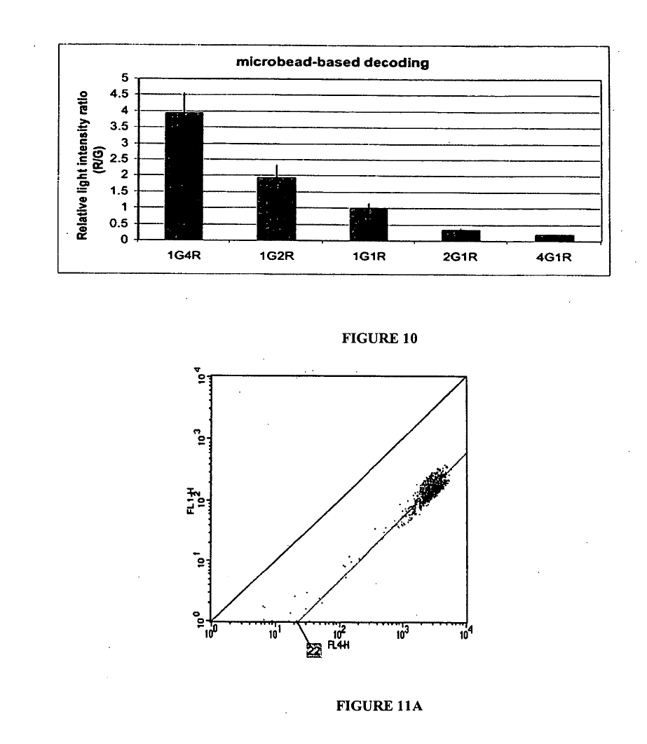

Since the color types and fluorophore numbers can be precisely controlled with the trimers or tetramers, the number of pseudocolors from the nanobarcodes imaging different probes is achieved with heretofore unachievable intensity and accuracy. Even with the naked eye, five different barcodes could be distinguished from FIG. 6B. The pseudocolor method was then used for qualitative detection. The sample mixture, which consisted of four target DNA from bacterial and viral pathogens respectively was analyzed with nanobarcodes, and four different pseudocolors were revealed from the overlay image as shown in FIG. 6C. Compared to FIG. 6B, it was found that four different nanobarcodes, 4G 1R, 2G1R, 1G1R, 1G4R, were bound to different microbeads as designed. With the pre-assigned barcode library (Table 5), it was determined that the sample contained DNA from four pathogens: Bacillus anthracis, Francisella tularensis, Ebola virus, and SARS Coronavirus. The quantitative decoding results at the population level (with thousands of microbeads) using different fluorescent intensity ratios are also shown in FIG. 10. Taken together, with one barcode(e.g. 1G1R) serving as a reference, other nanobarcodes are easily decoded. For example, if the intensity ratio between Red and Green fluorescence for a code is around 4, then the code will be 1G4R.

### Example 5

### Flow Cytometry

The application of nanobarcodes to multiplexed molecular detections was further demonstrated via flow cytometry. The fluorescence intensity ratio is the basis of decoding of any nanobarcodes aGbR. Under the same conditions, the fluorescence intensity ratio λ between each individual green (g) and red (r) dye is constant

$$( λ = \frac{g}{r} ) .$$

The green and red fluorescence intensities (G and R, respectively of a microbead bound with nanobarcodes are calculated by formulas G=n×(a×g) and R=n×(b×r), respectively.

The total fluorescence intensity ratio (K) between red and green fluorophores

$$( K = \frac{R}{G} )$$

can be used to calculate the code number,

$$\frac{a}{b} ,$$

using the formula

$$\frac{a}{b} = \frac{1}{K λ} .$$

When G and R are measured using flow cytometry, K can be calculated with the equation, log(G )=log(R)−log(K), where log (K) is the intercept of a two-color (green-red) flow plot. Thus the constant λ can be calculated with one known barcodes a reference (

$$\frac{{a}_{ref}}{{b}_{ref}}$$

is known) using the formula

$$\frac{1}{{K}_{ref}} \frac{{b}_{ref}}{{a}_{ref}} .$$

Once λ is determined, the code number of other nanobarcodes,

$$\frac{a}{b} ,$$

can be obtained with the equation;

$$\frac{a}{b} = \frac{1}{K λ} = \frac{{K}_{ref}}{K} \times \frac{{a}_{ref}}{{b}_{ref}} ,$$

where K is derived from the flow plot.

In this experiment, a known target DNA from Francisella tularensis (Sjostedt et al., “Detection of Francisella tularensis in Ulcers of Patients with Tularemia by PCR”. J. Clin. Microbiol. 35:1045-1048 (1997) which is hereby incorporated by reference in its entirety which was coded by 2G1R (i.e.,

$$( i . e . , \frac{{a}_{ref}}{{b}_{ref}} = 2 ) ,$$

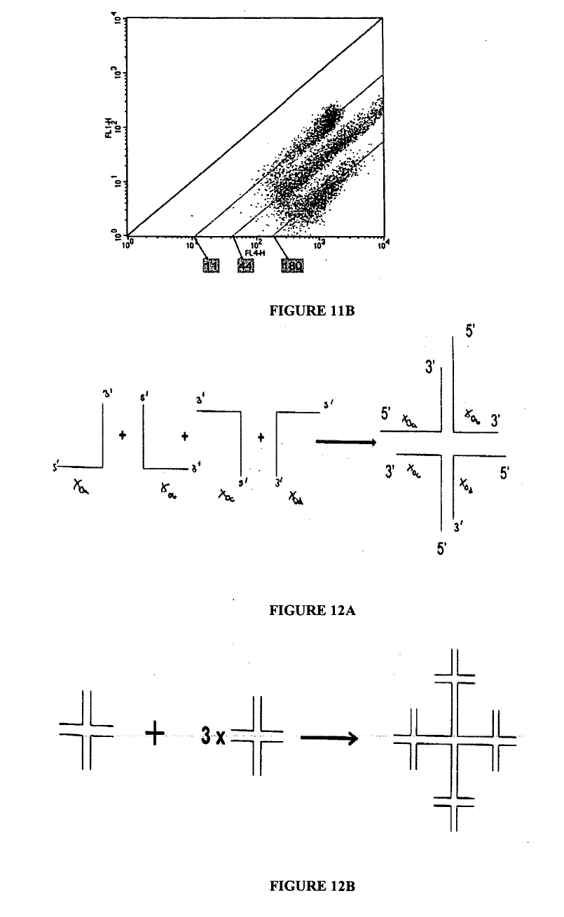

was used as a reference to determine λ, as shown in FIG. 11A. The measured value of K_{ref} (i.e. K_{2G1R}) from FIG. 11A was 22 and thus λ was equal to 1/44

$$( = \frac{1}{22} \times \frac{1}{2} ) .$$

Based on the equation,

$$\frac{a}{b} = \frac{1}{K λ} ,$$

the code number,

$$\frac{a}{b} ,$$

for any other nanobarcodes, was equal to 44/K. Since K can be measured for each barcode by the flow plot, the code number can be decoded.

To simultaneously detect multiple pathogens, a solution containing three pathogen DNA mixtures was formulated with lone irrelevant DNA as a control. This solution was treated as a sample with “unknown” pathogens. Samples were allowed to bind to the microbeads without saturation before the fluorescence intensity ratio was measured for each microbead via flow cytometry (FIG. 11B).

From the interceptions on the flow plots the values of K were determined, from the top line to the bottom line, as 11, 44, and 180, respectively. Thus the code numbers were calculated to be around 4.0, 1.0, and 0.25, respectively. Therefore, the detected nanobarcodes were 4G1R, 1G1R, and 1G4R. After referring to the pre-assigned barcode library (Table 5), it was concluded that the “unknown” sample contained DNA molecules from three pathogenic species; Bacillus anthracis, Ebola virus, and SARS-Coronavirus. The concentration of each pathogenic DNA was 62.5 pM, and only 10 μl of sample was used. Thus, the detection limit was 6.2×10⁻¹⁶ mole or 620 attomole. The detection was completed within 30 seconds. The success of simultaneously identifying multiple pathogens with an attomole sensitivity within a very short period of time (<1 min) demonstrated the great potential of nanobarcodes for multiplexed molecular detection.

### Example 6

### Dot Blotting Assay

Nanobarcodes can also be used for blotting-based detection (Southern, Northern, and Western). Dot blotting was used to demonstrate this potential. The dot blot assay was prepared by placing a drop (around 0.5 μl) of DNA solution (about 20 μM) including controls of a 15-mer oligonucleotide with irrelevant sequences and a 6.1 kb plasmid DNA onto a Zeta-probe membrane (Bio-Rad, Hercules, Calif.). After the membrane was air-dried DNA molecules on the membrane were cross-linked with a UV crosslinker (Strategene, San Diego, Calif.). The membrane was then pre-hybridized with Church buffer at 60° C. for 2 hours. Following pre-hybridization, the buffer was removed, and the membrane was submerged into a hybridization buffer (1×SSC buffer containing 1% SDS) containing nanbarcodes and incubated for overnight at 25° C. After hybridization, the membrane was evaluated with fluorescence microscopy.

Six samples, including the controls of 27-mer ssDNA with irrelevant sequences and a 6.1 kb plasmid DNA, were first blotted onto a membrane; nanobarcodes were then used to hybridize onto target DNA for detection (FIG. 7A). Simultaneous detections of four pseudocolors on the resultant membrane (FIG. 7B) indicated four DNA targets, which were subsequently identified by referring to a pre-assigned decoding library. As expected, no fluorescent signals were detected in the two control spots, suggesting a high specificity of the nanobarcode-based detection.

### Example 7

### Preparation of X-Shaped Nucleic Acid Trimer

The X-shaped DNA (X-DNA) sequences were designed, as set forth in Table 6. They were commercially synthesized (Integrated DNA Technologies).

TABLE 12

Sample Sequences of oligonucleotides that
construct X-DNA

| | | |
|---|---|---|
| Strand | Segment 1 | Segment 2 |

| | | |
|---|---|---|
| X₀₁ | 5′-p-ACGT | CGA CCG ATG AAT AGC GGT CAG ATC |
| | | CGT AC C TAC TCG-3′ |
| | | (SEQ ID NO:85) |
| X₀₂ | 5′-p-ACGT | CGA GTA GGT ACG GAT CTG CGT ATT |
| | | GCG AA C GAC TCG-3′ |
| | | (SEQ ID NO:86) |
| X₀₃ | 5′-p-ACGT | CGA GTC GTT CGC AAT ACG GCT GTA |
| | | CGT AT G GTC TCG-3′ |
| | | (SEQ ID NO:87) |
| X₀₄ | 5′-p-ACGT | CGA GAC CAT ACG TAC AGC ACC GCT |
| | | ATT CA T CGG TCG-3′ |
| | | (SEQ ID NO:88) |

Without further purification oligonucleotides were dissolved in annealing buffer (10 mM Tris, pH=8.0, 1 mM ethylenediaminetetraacetic acid (EDTA), aid 50 mM NaCl) with a final concentration of 50 mM. X-DNA was constructed by mixing four oligonucleotide components (with the same molar ratio for each oligonucleotides) in sterile Milli-Q water with a final concentration of 20 mM for each oligonucleotide, Hybridizations, were performed according to the following procedures: (i) denaturation at 95° C. for 2 min; (ii) cooling at 65° C. and incubation for 5 min.; (ii) annealing at 60° C. for 2 min; and (iv) further 0.5 min with a continuous temperature decrease at a rate of 1° C. per min. The annealing steps were repeated a total of 40 times. The final annealed products were stored at 4° C. The X₀₁ to X₀₄ were four corresponding single oligonucleotide chains that formed an X-DNA.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A detection molecule for detection of a target molecule, said detection molecule comprising:
a probe specific to the target molecule;
one or more multimer nucleic acid molecules connected to said probe; and
one or more label molecules linked to at lease one of said one or more multimer nucleic acid molecules.

2. The detection molecule of claim 1, wherein said one or more multimer is a trimer.

3. The detection molecule of claim 1, wherein said one or more multimer is a tetramer.

4. The detection molecule of claim 1, wherein said one or more multimer is Y-shaped.

5. The detection molecule of claim 1, wherein said one or more multi mer is X-shaped.

6. The detection molecule of claim 2, wherein each of said one or more trimers comprises a first, a second, and a third polynucleotide, wherein at least a portion of the first polynucleotide is complementary to at least a portion of the second polynucleotide wherein at least a portion of the first polynucleotide is complementary to at least a portion of the third polynucleotide, wherein at least a portion of the second polynucleotide is complementary to at least a portion of the third polynucleotide, and wherein the first, second, and third polynucleotides are associated together to form a trimer.

7. The detection molecule of claim 2, wherein the first, second, and third polynucleotides comprise a first region, a second region located 3′ to the first region, and a third region located 3′ to the second region, wherein the second region of the first polynucleotide comprises a region complementary to the third region of the third polynucleotide, the third region of the first polynucleotide comprises a region complementary to the second region of the second polynucleotide, and the third region of the second polynucleotide comprises a region complementary to the second region of the third polynucleotide.

8. The detection molecule of claim 1, wherein said one or multimer comprises nucleic acids having shapes selected from X-, Y-, T-shape or a combination thereof.

9. The detection molecule of claim 3, wherein each of said one or more trimers has three branches.

10. The detection molecule of claim 6, wherein at least one of the polynucleotides comprises a sticky end.

11. The detection molecule of claim 6, wherein the first, second, and third polynucleotides are DNA.

12. The detection molecule of claim 6 wherein each of said one or more trimers has a plurality of labels.

13. The detection molecule of claim 6, wherein each of said plurality of labels is coupled to a separate polynucleotide.

14. The detection molecule of claim 1, wherein the probe is a nucleic acid molecule complementary to a target nucleic acid molecule.

15. The detection molecule of claim 2, wherein the detection molecule comprises a plurality of trimers.

16. The detection molecule of claim 14, wherein said plurality of trimers comprises trimers having labels and trimers not having labels.

17. The detection molecule of claim 15, wherein said trimers not having labels are linked to said trimers not having labels.

18. The detection molecule of claim 1, wherein the label is selected from the group consisting: of fluorescent moieties, chemiluminescent moieties, and quantum dots.

19. A composition for the detection of one or more analytes comprising
a) a plurality of barcodes comprising a first probe capable of specifically binding to a first region on an analyte, one or more multimer nucleic acid molecules, and one or more labels, and
b) a plurality of solid supports comprising one or more second probes capable of specifically binding to a second region on an analyte.

20. The composition of claim 19, wherein said one or more analytes is selected from the group consisting of

21. The composition of claim 19, wherein said one or more labels is selected from the group consisting chromophores, fluorescent groups, electrochemical moieties, enzymes, radioactive moieties, chemiluminescent moieties, quantum dots and: a combination thereof.

22. The composition of claim 19, wherein each pair of barcodes and solid supports is capable of detecting a different analyte.

23. The composition of claim 19, wherein said first and second probes are directly or indirectly linked to said solid supports or barcodes.

24. The composition of claim 23, wherein said indirect link-age comprises avidin and biotin.

25. A method of detecting whether a target molecule is present in a sample, comprising:
providing a detection molecule comprising;
a probe specific to said target molecule;
one or more multimer nucleic acid molecules linked to the probe;
one or more label molecules linked to said one or more multimer nucleic acid molecule;
contacting sample components with the detection molecule under conditions effective to permit said target molecule to specifically bind to said probe;
identifying any specific binding of said target molecule to the probe; and
thereby detecting the presence of said target molecule in the sample.

26. The method of claim 25, wherein said one or more multimer is a trimer.

27. The method of claim 25, wherein said one or more multimer is a tetramer.

28. The method of claim 25, wherein said one or more multimer comprises Y-shaped nucleic acid molecules.

29. The method of claim 25, wherein said one or more multimer comprises X-shaped nucleic acid molecules.

30. The method of claim 25, wherein said one or more multimer comprises nucleic acids having shapes selected from X-, Y-, T-shape or a combination thereof.

31. The method of claim 26, wherein said one or more trimer comprises a first, a second, and a third polynucleotide, wherein at least a portion of the first polynucleotide is complementary to at least a portion of the second polynucleotide, wherein at least a portion of the first polynucleotide is complementary to at least a portion of the third polynucleotide, wherein at least a portion of the second polynucleotide is complementary to at least a portion of the third polynucleotide, and wherein the first, second, and third polynucleotides are associated together to form a trimer.

32. The method of claim 31, wherein the first, second, and third polynucleotides comprise a first region, a second region located 3′ to the first region, and a third region located 3′ to the second region, wherein the second region of the first polynucleotide comprises a region complementary to the third region of the third polynucleotide, the third region of the first polynucleotide comprises a region complementary to the second region of the second polynucleotide and the third region of the second polynucleotide comprises a region complementary to the second region of the third polynucleotide.

33. The method of claim 31, wherein the first, second, and third polynucleotides are DNA.

34. The method of claim 31, wherein each of said one or more trimer has a plurality of labels.

35. The method of claim 25, wherein the probe is selected from a group consisting of a nucleic acid, a peptide, a protein, an antibody, a member of a specific binding-pair and an aptamer.

36. The method of claim 25, wherein said one or more label molecule is selected from the group consisting of chromophores, electrochemical moieties, enzymes, radioactive moieties, phosphorescent groups, fluorescent moieties, chemiluminescent moieties, quantum dot and a combination thereof.

37. The method of claim 25, wherein said one or more label is a fluorescent label.

38. The method of claim 25, wherein said multimer comprises at least two different fluorescent labels.

39. The method of claim 25, further comprising capturing the target molecule to a solid support.

40. The method of claim 25, wherein said contacting comprises providing a solid support linked to one or more capture probes specific to the target molecule and contacting the sample with said solid support either before or after contacting with said detection molecule.

41. A method of detecting whether one or more target molecules is present in a sample, comprising:
providing a plurality of different detection molecules each comprising;
a probe specific to a different target molecule;
one or more multimer nucleic acid molecules linked to said probe;
one or more label molecules linked to said one or more multimer nucleic acid molecule;
contacting sample components with the plurality, of detection molecules under conditions effective to permit said one or more target molecules to specifically bind said probe;
identifying any specific binding of said different target molecule to said probe;
thereby detecting the presence of said one or more target molecules in the sample.

42. A method for determining whether one or more analyte is present in a sample comprising:
a. contacting said sample with a plurality of barcodes- wherein each of said plurality of barcodes comprise a plurality of multimers) one or more labels each emitting a different detectable signal, and a probe specific for different analytes;
b. detecting said detectable signal(s);
c. measuring a ratio of signal intensities produced by at least two of said labels; and
thereby determining one or more analyte is present based on the measured ratio.

43. The method of claim 36, wherein said detecting is by flow cytometry.

44. The method of claim 36, wherein said labels are fluorescent labels.

45. The method of claim 36, wherein each barcode comprises a different number and/or different types of labels permitting a distinction between different analytes.

46. The method of claim 36, wherein each of said barcodes comprises only two different fluorescent labels.
